SEQUENCE LISTING (27 sequences)

SEQ ID NO: 1 - DNA, 22 bp, Artificial Sequence
  Synthetic
gatcacacgcgtcaaggtggcc

SEQ ID NO: 2 - DNA, 24 bp, Artificial Sequence
  Synthetic
ggtctcgatgcacgatcatgtgcc

SEQ ID NO: 3 - DNA, 33 bp, Artificial Sequence
  Synthetic
gtcaaggtggccgtcaggatactcaatcgtcag

SEQ ID NO: 4 - DNA, 33 bp, Artificial Sequence
  Synthetic
ctgacgattgagtatcctgacggccaccttgac

SEQ ID NO: 5 - DNA, 27 bp, Artificial Sequence
  Synthetic
cgagttcctgcgcgcctcgtgcggctc

SEQ ID NO: 6 - DNA, 27 bp, Artificial Sequence
  Synthetic
gagccgcacgaggcgcgcaggaactcg

SEQ ID NO: 7 - DNA, 30 bp, Artificial Sequence
  Synthetic
gcgagttcctgcgcgactcgtgcggctcgc

SEQ ID NO: 8 - DNA, 30 bp, Artificial Sequence
  Synthetic
gcgagccgcacgagtcgcgcaggaactcgc

SEQ ID NO: 9 - DNA, 30 bp, Artificial Sequence
  Synthetic
cctcagcgtgcagcatccaatgtgtttgct

SEQ ID NO: 10 - DNA, 30 bp, Artificial Sequence
  Synthetic
agcaaacacattggatgctgcacgctgagg

SEQ ID NO: 11 - DNA, 30 bp, Artificial Sequence
  Synthetic
cctcagcgtgcaacagccaatgtgtttgct

SEQ ID NO: 12 - DNA, 30 bp, Artificial Sequence
  Synthetic
agcaaacacattggctgttgcacgctgagg

SEQ ID NO: 13 - DNA, 30 bp, Artificial Sequence
  Synthetic
cctcagcgtgcagcagccaatgtgtttgct

SEQ ID NO: 14 - DNA, 30 bp, Artificial Sequence
  Synthetic
agcaaacacattggctgctgcacgctgagg

SEQ ID NO: 15 - DNA, 36 bp, Artificial Sequence
  Synthetic
cgccctcagcgtgcagaagaaaatgtgtttgctatg

SEQ ID NO: 16 - DNA, 36 bp, Artificial Sequence
  Synthetic
catagcaaacacattttcttctgcacgctgagggcg

SEQ ID NO: 17 - DNA, 36 bp, Artificial Sequence
  Synthetic
cgcgcccaacgcgccgccgccaatgtgttcgccatg

SEQ ID NO: 18 - DNA, 36 bp, Artificial Sequence
  Synthetic
catggcgaacacattggcggcggcgcgttgggcgcg

SEQ ID NO: 19 - DNA, 20 bp, Artificial Sequence
  Synthetic
taatacgactcactataggg

SEQ ID NO: 20 - DNA, 15 bp, Artificial Sequence
  Synthetic
ttcggcaaggtgaag

SEQ ID NO: 21 - DNA, 15 bp, Artificial Sequence
  Synthetic
cacttgcagcatctg

SEQ ID NO: 22 - DNA, 20 bp, Artificial Sequence
  Synthetic
ggatctgttcgcacctgacg

SEQ ID NO: 23 - DNA, 17 bp, Artificial Sequence
  Synthetic
ctgccgcttggcgggcg

SEQ ID NO: 24 - DNA, 20 bp, Artificial Sequence
  Synthetic
cgtaccactggcatcgtgat

SEQ ID NO: 25 - DNA, 20 bp, Artificial Sequence
  Synthetic
gtgttggcgtacaggtcttt

SEQ ID NO: 26 - DNA, 24 bp, Artificial Sequence
  Synthetic
ctcagttgaatccaacaagtctac

SEQ ID NO: 27 - DNA, 25 bp, Artificial Sequence
  Synthetic
ctccacttgcatattccatgattag

(19) United States
(12) Patent Application Publication
(10) Pub. No.: US 2009/0042810 A1
(43) Pub. Date: Feb. 12, 2009

(54) AMPK Deficient Animals, Screening Methods, And Related Therapeutics And Diagnostics

(75) Inventor: Jongkyeong Chung, Yusong-Gu Taejon (KR)

Correspondence Address: MEDLEN & CARROLL, LLP, 101 HOWARD STREET, SUITE 350, SAN FRANCISCO, CA, 94105, US

(73) Assignee: GenExel-Sein, Inc.

(21) Appl. No.: 12/107,448

(22) Filed: Apr. 22, 2008

Related U.S. Application Data
(60) Provisional application No. 60/926,480, filed on Apr. 27, 2007.

(51) Int. Cl. A61K 31/70; A01K 67/027; A61P 1/00; C12Q 1/48

(52) U.S. Cl. 514/23; 800/9; 800/3; 435/15

(57) ABSTRACT

The invention provides transgenic Drosophila embryos comprising in the germ cells an adenosine monophosphate-activated protein kinase (AMPK) null mutation. The transgenic Drosophila embryos are useful in screening drug candidates for treatment of a disease, such as cancer, kidney disease, diabetes, intestinal disease, and obesity. The invention further provides methods for detecting disease in a tissue, comprising detecting a change in AMPK activity in the tissue compared to a control tissue. Also provided are methods for reducing symptoms of a disease in a subject, comprising administering a therapeutic amount of a drug that changes AMPK activity to the subject.

This application claims priority to co-pending U.S. Provisional Application Ser. No. 60/926,480, filed Apr. 27, 2007, herein incorporated by reference in its entirety.

### SUMMARY OF THE INVENTION

The invention provides, in one embodiment, a transgenic Drosophila embryo comprising in its germ cells an adenosine monophosphate-activated protein kinase (AMPK) null mutation on one or both AMPK alleles, wherein the mutation results in the embryo exhibiting, compared to a Drosophila embryo lacking the null mutation, at least one phenotype selected from a) increase in number of embryos that do not develop into larvae, b) change in cuticle structure, c) decrease in number of ventral denticle belts, d) change in organization of epidermis tissue, e) change in epithelial cell polarity, f) decrease in number of embryos forming a cuticle, g) decrease in level of expression around an epithelial basolateral surface of at least one of apical complex marker and of β-catenin, h) increase in number of unpolarized round epithelial cells lacking contact with underlying tissue, i) increase in number of ectopic actin structures in a basolateral region of a wing disc, j) increase in nuclear size, k) change in metaphase chromosome alignment, l) increase in lagging chromosomes during anaphase, m) increase in chromosomal polyploidy in a cell, and n) increase in chromosome content in a brain neuroblast cell. In a particular embodiment, the cell having chromosomal polyploidy comprises a brain neuroblast cell. In a further embodiment, the epithelial cells of the embryo comprise reduced levels of phosphorylated non-muscle myosin regulatory light chain (MRLC) compared to a Drosophila embryo lacking the null mutation. In an alternative embodiment, increased expression of non-muscle myosin regulatory light chain (MRLC) in the embryo results in at least one phenotype selected from a) reversal of the change in the epithelial cell polarity, b) increase in number of embryos that form a cuticle, and c) decrease in chromosomal polyploidy.

While not intending to limit the method of producing the invention's transgenic embryos, in one embodiment, the embryo is generated by mating a male transgenic Drosophila and a female transgenic Drosophila each bearing one artificially mutated AMPK allele in its germ cells. In a particular embodiment, the artificially mutated AMPK allele is generated by an autosomal flipase recombination target dominant female sterile (FLP-DFS) method.

The invention also provides a method for screening a drug candidate for treatment of a disease comprising a) providing i) transgenic Drosophila embryo as described herein, and ii) a drug candidate, b) administering the drug candidate to the embryo, and c) determining the embryo's response to the drug candidate. While not intending to limit the type of response that is determined following treatment with the drug candidate, in one embodiment, the response comprises a change in at least one of the phenotypes disclosed herein. Without limiting the type of disease, in one embodiment, the disease is selected from cancer, kidney disease, diabetes, intestinal disease, and obesity, and any disease that comprises increased body weight of the subject.

In some embodiments, the drug candidate is an AMPK-activating drug, as exemplified by, but not limited to a drug comprising one or more of metformin (N,N-dimethylimidodicarbonimidic diamide hydrochloride), AICAR (5-aminoimidazole-4-carboxamide-1-β-D-ribofuranoside), resveratrol (trans-3,4′,5-trihydroxystilbene), and a thiazolidinedione compound. The AMPK-activating drugs may be screened for their efficacy in treating any disease including, without limitation, cancer, kidney disease, diabetes and intestinal disease. In one embodiment the intestinal disease is characterized by reduced nutrient absorption by intestines. Alternatively, or in addition, the intestinal disease is caused by an organism that alters intestinal epithelial cell polarity, as exemplified by Salmonella typhimurium.

In alternative embodiments, the drug candidate is an AMPK-inhibiting drug, as exemplified by, but not limited to, compound C. While not intending to limit the type of disease, in one embodiment, the AMPK-inhibiting drug may be screened for its efficacy in treating kidney disease, and diseases that comprise increased body weight, such as obesity.

The invention additionally provides a method for detecting a disease in a tissue, comprising detecting a change in AMPK activity in the tissue compared to a control tissue. In one embodiment, the disease is selected from cancer, kidney disease, diabetes, and intestinal disease.

Also provided herein is a method for reducing symptoms of a disease in a subject, comprising administering a therapeutic amount of a drug that changes (i.e., increases or reduces) AMPK activity to the subject. It may be desirable, though not necessary, to further determine a reduction in symptoms of the disease.

In one embodiment, the change in AMPK activity is a reduction in AMPK activity, and the disease is selected from kidney disease and increased body weight. In another embodiment, the change in AMPK activity is an increase in AMPK activity, and the disease is selected from kidney disease and intestinal disease.

### BRIEF DESCRIPTION OF THE DRAWINGS

FIG. 1 shows that AMPK is essential for normal development. a, Schematic genomic organization and restriction map of AMPK locus. BHI, BamHI; ERV, EcoRV. b, Southern blot analyses of AMPK in wild type (WT) and heterozygous AMPK mutants (AMPK^{D1}/+, AMPK^{D2}+). c, RT-PCR analyses of AMPK in wild type (WT) and AMPK-null mutants (AMPK^{D1}, AMPK^{D2}). rp49 was used as control. d, Immunoblot analysis of AMPK in embryos (E), larvae (L), pupae (P), adult females (F) and adult males (M) of wild type (WT), and larvae of AMPK^{D1} and AMPK^{D2}. Tubulin (Tub) was used as control. e, Restored viability of AMPK-null (AMPK^{D1}) mutants by transgenic expression of wild-type (AMPK^{WT}) but not by kinase-dead (AMPK^{KR}) AMPK. tub-Gal4/+ was used as control (WT). f, Survival rates of wild type (WT) and AMPK-null mutants (AMPK^{D1}) during development: 1^{st}, 2^{nd} and 3^{rd} instar larva, pupa (P), and adult (A). g, h, Cuticles (g) and surfaces (h) of wild-type (WT) and AMPK-GLC (AMPK^{D1}) embryos visualized by dark field (DF), phase contrast (PC), or scanning electron microscopy (SEM). Scale bars: yellow, 50 μm; white, 20 μm.

FIG. 2 shows that AMPK and its upstream kinase LKB1 are required for epithelial and genomic integrity. a-c, Epithelia of wild-type (WT), AMPK-GLC (AMPK^{D1}), and LKB1-GLC (LKB1^{X5}) embryos stained with anti-Baz (green) and anti-Dlg (red) antibodies and Hoechst 33258 (DNA, blue). d, Wild-type (WT), AMPK-GLC (AMPK^{D1}), and LKB1-GLC (LKB1^{X5}) embryos stained with Hoechst 33258 (DNA, blue) and anti-actin antibody (Act, red). Arrows indicate cells with an excessive DNA content. e, Aceto-orcein staining of wild-type (WT), AMPK-null (AMPK^{D1}), and LKB1-null (LKB1^{X5}) larval neuroblasts. f-m, Wing discs (f-i) and brain hemispheres d-m) of wild-type (WT), LKB1-null (LKB1^{X5}), LKB1-null expressing AMPK^{TD} (LKB1^{X5}, hs>AMPK^{TD}) and LKB1-null expressing MRLC^{EE} (LKB1^{X5}, MRLC^{EE}) larvae stained with anti-aPKC (apical complex marker⁵, green) and anti-Dlg (red) antibodies and Hoechst 33258 (DNA, blue) (f-i), or with anti-PH3 antibody (j-m). Vertical images were obtained by optical sectioning. Arrows indicate mitotic chromosomes with extreme polyploidy. Scale bars: yellow, 50 μm; white, 10 μm; orange, 5 μm.

FIG. 3 shows AMPK phosphorylates MRLC to regulate the mitosis and cell polarity. a-d, Phosphorylation of MRLC by AMPK. a, b, AMPK assay using human MRLC (GST-MRLC) substrate, with (+) or without (−) AMP. The phosphorylation was visualized by autoradiography (³²P-MRLC) or anti-phospho MRLC immunoblot (pMRLC), and total MRLC by coomassie staining (a) or anti-GST immunoblot (b). c, Measurement of AMPK activity using the following substrates: human ACC2 (gray circle, GST-ACC2), wild-type (black square, GST-MRLC WT) and Thr18Ala/Ser19Ala-mutant (black cross, GST-MRLC^{AA}) human MRLC. Error bars indicate s.d. of two experiments. [S], substrate concentration; N/D, not determined. d, AMPK assay using wild-type (WT) and Thr18Ala/Ser19Ala-mutant (AA) GST-MRLC substrate. CC and ML-7 were treated as indicated. Phosphorylated MRLC was visualized by autoradiography (³²P-MRLC), and total MRLC by coomassie staining. e, Cuticles and epithelial structures of wild-type (WT), AMPK-GLC (AMPK D) and AMPK-GLC expressing MRLC^{EE} (AMPK^{D1}, MRLC^{EE}) embryos visualized by dark-field (DF) microscopy, anti-aPKC (green) and anti-Dlg (red) antibody staining, and Hoechst 33258 staining (DNA, blue). f, Quantification of cuticle phenotypes in AMPK-GLC (AMPK^{D1}) and AMPK-GLC expressing MRLC^{EE} (AMPK^{D1}, MRLC^{EE}) embryos (*, P=1.91×10⁻³). Error bars indicate s.d. of three experiments. g, The brain hemispheres from AMPK-null (AMPK^{D1}) and AMPK-null expressing MRLC^{EE} (AMPK^{D1}, MRLC^{EE}) larvae stained with anti-PH3 antibody. Arrows indicate mitotic chromosomes with extreme polyploidy. h, Quantification of genomic polyploidy in mitotic neuroblasts (*, P=1.36×10⁻²; P=5.32×10⁻³; ***, P=2.68×10⁻²). Error bars indicate s.d. of more than three experiments. Scale bars: yellow, 50 μm; white, 10 μm.

FIG. 4 shows energy deprivation induces polarization of LS174T cells via MRLC phosphorylation by AMPK. a-d, Polarization of LS174T cells by energy deprivation. After 2-hr treatment of 2DG, LS174T cells were stained with anti-tubulin (Tub, green in a), anti-villin (Vil, green in b), anti-CD66/CEA (CD66, green in c), or anti-CD71/transferrin (CD71, green in d) antibodies, and TRITC-phalloidin (Act, red). Polarized markers are indicated by arrows. e, 2DG-induced phosphorylation of MRLC by AMPK. LS174T cells were stained with anti-phospho MRLC antibody (pMRLC, green) and TRITC-phalloidin (Act, red), after indicated treatments. f, Inhibition of the 2DG-induced polarization by human AMPK^{DN}. After 2-hr treatment of 2DG, cells were stained with TRITC-phalloidin (Act, red). AMPK^{DN} was detected by HA-tag (green). g, Knockdown of MRLC suppressed the 2DG-induced polarization. After 2-hr treatment of 2DG, MRLC (siMRLC) or control (siControl) siRNA-treated cells were stained with TRITC-phalloidin (Act, red). h, Polarization of LS174T cells by expression of MRLC^{EE}. After 24 hr of transfection, cells were stained with TRITC-phalloidin (Act, red). MRLC^{EE} detected by GFP-tag (green) was co-localized with polarized actin structures (arrow).

FIG. 5 shows identification and characterization of Drosophila AMPK. a, Amino acid sequence similarities (%) of Drosophila AMPK with its human and yeast (S. cerevisiae) homologues. b, Activatory phosphorylation (pAMPK) of AMPK in the S2 cell. Energy deprivation was induced by 2-deoxyglucose (2DG) treatment. LKB1 dsRNA (L), and AMPK dsRNA (A) were treated as indicated. Tubulin (Tub) was used as control.

FIG. 6 shows the epithelial structure is disorganized in AMPK-GLC embryos. a, wild-type (WT) and AMPK-GLC (AMPKD1) embryos were stained with anti-armadillo (Arm, green) antibody. DNA was visualized by Hoechst 33258 (blue). b, wild-type (WT) and AMPKGLC (AMPKD1) embryos were stained with anti-actin (Act, green) antibody. DNA was visualized by Hoechst 33258 (blue). Scale bars: 5 μm.

FIG. 7 shows the polarity defects of AMPK-GLC epithelia were not restored by p35 expression, while apoptotic cell death was suppressed. a-c, Epithelial organization of wildtype (a, WT), AMPK-GLC (b, AMPKD1), and p35-expressing AMPK-GLC (c, AMPKD1, hs>p35) embryos was visualized by anti-aPKC (green, converted from Cy5 signal) and anti-Dlg (red, converted from FITC signal) antibody staining. DNA was visualized by Hoechst 33258 (blue), and apoptotic cells (marked by arrowheads) were visualized by TUNEL staining (red, right panels). Arrows indicate non-apoptotic cells with aberrant aPKC staining in the basolateral surface. d-f, Analyses of epithelial structure in p35-expressing AMPK-GLC embryos (AMPKD1, hs>p35) with various polarity markers. The embryos were stained with anti-aPKC (green, converted from Cy5 signal), anti-Baz (green, converted from Cy5 signal), anti-Arm (green, FITC signal) and anti-Dlg (red, converted from FITC signal) antibodies. The absence of apoptosis was confirmed by TUNEL staining (red, bottom panels), and DNA was visualized by Hoechst 33258 (blue). g. Quantification of epithelial cell apoptosis in AMPKGLC (AMPKD1) and p35-expressing AMPK-GLC (AMPKD1, hs>p35) embryos (*, P=2.22×10−5). Error bars indicate s.d. of three experiments. Scale bar: 10 μm.

FIG. 8 shows actin structure is disorganized in LKB1- and AMPK-null mutants. Wild-type (a, WT), AMPK-null (b, AMPKD1), LKB1-null (c, LKB1X5), and LKB1-null expressing AMPKTD (d, LKB1X5, hs>AMPKTD) wing discs were stained with TRITC-phalloidin (Act, red) and Hoechst 33258 (DNA, blue). Vertical images were obtained by optical sectioning. Scale bar: 10 μm.

FIG. 9 shows mitotic defects in AMPK- and LKB1-null embryos are similar. a, AMPK- and LKB1-null embryos showed defects in metaphase chromosome alignment (arrowheads). Wild-type (WT), AMPK-GLC (AMPKD1), and LKB1-null (LKB1X5) embryos were stained with anti-PH3 (red) and anti-tubulin (Tub, green) antibodies. b, AMPK- and LKB1-null embryos showed lagging chromosomes during anaphase (arrowheads). Wild-type (WT), AMPK-GLC (AMPKD1), and LKB1-null (LKB1X5) embryos were stained with anti-PH3 (red) antibody. c, AMPK- and LKB1-null embryos contained mitotic cells with polyploidy (arrows). AMPK-GLC (AMPKD1), and LKB1-GLC (LKB1X5) embryos were stained with anti-PH3 (green) and anti-actin (Act, red) antibodies. Magnified images of polyploid chromosomes are presented as insets. Scale bars: 5 μm.

FIG. 10 shows cuticle defects and reduced MRLC phosphorylation of LKB1-GLC embryos. a, Microscopic observation of wild-type (WT) and LKB1-GLC (LKB1X5) embryos. Dark field microscopy (DF) was used to visualize the cuticles of the embryos. b, Phosphorylation level of MRLC in wild-type (WT) and LKB1-GLC (LKB1X5) embryonic epithelia. Embryos were stained with anti-phospho MRLC (pMRLC, green) and anti-actin (Act, red) antibodies. DNA was visualized by Hoechst 33258 (blue). c, Protein expression levels of MRLC in wild-type (WT) and LKB1-GLC (LKB1X5) embryonic epithelia. Embryos were stained with anti-MRLC (MRLC, green) and anti-actin (Act, red) antibodies. DNA was visualized by Hoechst 33258 (blue). Scale bars: yellow, 50 μm; white, 10 μm.

FIG. 11 shows the polarity defects of LKB1-null wing disc epithelia were rescued by the expression of AMPKTD and MRLC^{EE}. Wing discs of wild-type (WT), LKB1-null (LKB1X5), AMPKTD-expressing LKB1-null (LKB1X5, AMPKTD), and MRLCEE-expressing LKB1-null (LKB1X5, MRLCEE) were stained with antibodies to various polarity markers including Crumbs (Crb, apical marker), E-cadherin (DCAD, adherens junction marker), Scribble (Scrib, basolateral marker), Bazooka (Baz, apical complex marker), and Na+/K+ ATPase (NaK, basolateral marker). DNA was visualized by Hoechst 33258. Vertical images were obtained by optical sectioning. Scale bars: 10 μm.

FIG. 12 shows AMPK phosphorylates the regulatory site of MRLC. a, Phosphorylation stoichiometry of wild-type (MRLCWT) and Thr18Ala/Ser19Ala-mutant (MRLCAA) human MRLC by AMPK holoenzyme purified from rat liver, expressed as mol phosphate per mol substrate. Error bars indicate s.d. of two independent experiments b, AMPK phosphorylated human MRLC primarily at Ser19, and to a lesser extent at Thr18. AMPK holoenzyme purified from rat liver was assayed using wild-type (WT), Thr18Ala (TA). Ser19Ala (SA), and Thr18Ala/Ser19Ala (AA) mutant forms of human MRLC (GST-MRLC). The phosphorylated MRLC (32P-MRLC) was visualized by autoradiography, and MRLC protein levels (GST-MRLC) were compared by coomassie gel staining.

FIG. 13 shows both human and Drosophila forms of AMPK are able to phosphorylate MRLC from each of the respective species. a,b, The kinase activities of wildtype (dAMPKWT), kinase-dead (dAMPKKR) and Thr184Ala mutant (dAMPKTA, Thr184 is the LKB1 phosphorylation site of dAMPK) forms of Drosophila AMPK, and wild-type (hAMPKWT) and kinase-dead (hAMPKDN) forms of human AMPK, which were purified by immunoprecipitation. These various forms of AMPK were assayed using MRLC substrates (GST-MRLC) from each of the respective species. The phosphorylated MRLC was visualized by autoradiography (32P-MRLC) or anti-phospho MRLC immunoblot (pMRLC). MRLC protein levels (GST-MRLC) were visualized by coomassie gel staining (a) or anti-GST immunoblot (b). AMPK protein levels (HA-AMPK) were visualized by anti-HA immunoblot. c,d, Drosophila AMPK (dAMPKWT) and human AMPK (hAMPKWT) purified by immunoprecipitation were assayed using wild-type (WT) and Thr21Ala/Ser22Ala mutant (AA) forms of Drosophila MRLC (GST-dMRLC), and wild-type (WT) and Thr18Ala/Ser19Ala mutant (AA) forms of human MRLC (GST-hMRLC), respectively. The phosphorylated MRLC (32P-MRLC) was visualized by autoradiography, and MRLC protein levels (GST-MRLC) were detected by coomassie gel staining.

FIG. 14 shows further verification of MRLC phosphorylation by AMPK. a, Drosophila (dAMPK) and human (hAMPK) AMPK purified by immunopreciptation were analyzed by silver staining. *, catalytic subunits of AMPK; **, antibody bands. b, AMPK phosphorylation of MRLC was specifically inhibited by AMPK inhibitor CC, but not by MLCK inhibitor ML-7. Conversely, MLCK phosphorylation of MRLC was specifically inhibited by ML-7, but not by CC. Drosophila AMPK (dAMPK), human AMPK (hAMPK), and human MLCK (MLCK) were assayed using MRLC substrates from each of the respective species. Drugs were directly added to the reaction solutions as indicated. The phosphorylated MRLC (32P-MRLC) was visualized by autoradiography, and MRLC protein levels (GSTMRLC) were visualized by coomassie gel staining. c, The catalytic subunit of human AMPK (hAMPKα) purchased from Cell Signaling was assayed using human MRLC as a substrate. CC was directly added to the reaction solutions as indicated. The phosphorylated MRLC (32PMRLC) was visualized by autoradiography, and MRLC protein levels (GST-MRLC) were determined by coomassie gel staining.

FIG. 15 shows MRLC phosphorylation was dramatically reduced in AMPK-GLC epithelia. a-d, Wild-type (a, WT), AMPK-GLC (b, AMPKD1), AMPK-GLC expressing AMPK (c, AMPKD1, tub>AMPKWT), and AMPK-GLC expressing LKB1 (d, AMPKD1, hs>LKB1) embryonic epithelia were stained with anti-phospho MRLC (PMRLC, green) and anti-actin (Act, red) antibodies and Hoechst 33258 (DNA, blue). e, f, Protein levels of MRLC are similar between wild-type and AMPK-GLC embryos. Wild-type (e, WT) and AMPK-GLC (f, AMPKD1) epithelia were stained with anti-MRLC (MRLC, green) and anti-actin (Act, red) antibodies. DNA was visualized by Hoechst 33258 (blue). Scale bar: 10 μm.

FIG. 16 shows energy deprivation induces MRLC phosphorylation via AMPK and LKB1 in Drosophila S2 cells. Phosphorylation of endogenous MRLC (pMRLC), and endogenous protein levels of MRLC (MRLC), tubulin (Tub), and Par-1 (Par-1) were analyzed by immunoblot analyses. 2DG and dsRNAs to white, MRLC, AMPK, Par-1 and LKB1 were treated as indicated.

FIG. 17 shows epithelial polarity defects of spaghetti-squash1 (sqh1) mutant. Wing imaginal discs of wild type (a, WT) and sqh1 (b) were stained with antibodies to aPKC (red, apical complex marker) and Dlg (green, basolateral marker). DNA was visualized by Hoechst 33258 (blue). Vertical images were obtained by optical sectioning. Scale bars: 10 μm.

FIG. 18 shows LKB1-AMPK signaling is functional in LS174T cells. a. Expression of LKB1 in mammalian cells. The protein expression of LKB1 in LS174T, HEK293T, and HeLa cell lines were analyzed by anti-human LKB1 (LKB1) immunoblot. The lack of LKB1 expression in HeLa was previously reported S9. Actin (Act) was used as a loading control. b, Energy deprivation-induced activation of AMPK in LS174T cells. Activatory phosphorylation of AMPK (pAMPK) was determined by immunoblot analyses after 2DG treatment for the indicated time period. Phosphorylation-induced gel shifts were detectable in anti-human AMPK (AMPK) immunoblot.

FIG. 19 shows energy deprivation induces the formation of brush border-like structures in LS174T cell line. Scanning electron micrograph (SEM) and transmission electron micrograph (TEM) of Control (a) and 2DG-treated (b) LS174T cells. In b, boxed regions, which indicate cell regions containing dense patches of microvilli, were magnified in bottom panels. Scale bars: 2 μm.

FIG. 20 shows energy deprivation-induced polarization of LS174T cells were mediated via MRLC phosphorylation by AMPK. a, 2DG-induced polarization of LS174T cells were inhibited by CC treatment. 2DG was treated for 2 hr. CC was treated as described in Supplementary Methods. Actin was visualized by TRITC-phalloidin staining (Act, red). b, Inhibition of Par-1 by siRNA (siPar-1) or Par-1DN did not affect the 2DG-induced polarization. After 2-hr treatment of 2DG, cells were stained with TRITC-phalloidin (Act, red). Par-1DN was detected by HA-tag (green). c, Confirmation of Par-1DN functionality. Par-1 DN inhibited the Wnt-induced activation of the TCF/LEF reporter, as previously described S6. HEK293T cells were transiently transfected with TOPFLASH and pRL-TK Renilla reporter plasmids. Wnt1 and Par-1DN plasmids were cotransfected as indicated. Dual luciferase assays were performed. Error bars indicate s.d. of two independent experiments. d, Confirmation of siRNA-mediated inhibition of Par-1. RT-PCR was used to visualize the amount of Par-1 and actin (Act) transcript, and western blot was used to visualize the amount of Par-1 and tubulin (Tub) protein, within the cells treated with control (siControl) or Par-1 (siPar-1) siRNA. e, Confirmation of siRNA-mediated inhibition of MRLC. Western blot was used to visualize the amount of MRLC and tubulin (Tub) within the cells treated with control (siControl) or MRLC (siMRLC) siRNA. f-h, Actin polarization (%) of LS174T cells with various treatments. 2DG was treated for 2 hr. Total numbers of cells counted are: 244 (Control), 216 (2DG), 111 (CC+2DG), 70 (2DG+AMPKDN), 83 (2DG+Par-1DN), 71 (2DG+siControl), 64 (2DG+siMRLC), 83 (2DG+siPar-1), 206 (GFP), and 102 (MRLCEE-GFP).

### DESCRIPTION OF THE INVENTION AND EXPERIMENTAL

AMP-activated protein kinase (AMPK) has been primarily studied as a metabolic regulator that is activated in response to energy deprivation (Kahn et al., Cell Metab. 1, 15-25 (2005)). Although there is relatively ample information on the biochemical characteristics of AMPK, not enough data exist on the in vivo function of the kinase. Here, using the Drosophila model system, we generated the first animal model with no AMPK activity and discovered novel physiological functions of the kinase. Surprisingly, AMPK-null mutants were lethal with severe abnormalities in cell polarity and mitosis, similar to those of LKB1-null mutants. Constitutive activation of AMPK restored many of the phenotypes of LKB1-null mutants, suggesting that AMPK mediates the polarity- and mitosis-controlling functions of LKB1. Interestingly, the regulatory site of non-muscle myosin regulatory light chain (MRLC) (Matsumura, Trends Cell Biol. 15, 371-377 (2005); Jordan et al., J. Cell Biol. 139, 1805-1819 (1997)) was directly phosphorylated by AMPK. Moreover, the phosphomimetic mutant of MRLC (Jordan et al., J. Cell Biol. 139, 1805-1819 (1997)) rescued the AMPK-null defects in cell polarity and mitosis, suggesting MRLC to be a critical downstream target of AMPK. Furthermore, the activation of AMPK by energy deprivation was sufficient to cause dramatic changes in cell shape, inducing complete polarization and brush border formation in the human LS174T cell line, through the phosphorylation of MRLC. In sum, our results demonstrate that AMPK plays highly conserved roles across metazoan species not only in the control of metabolism, but also in the regulation of cellular structures.

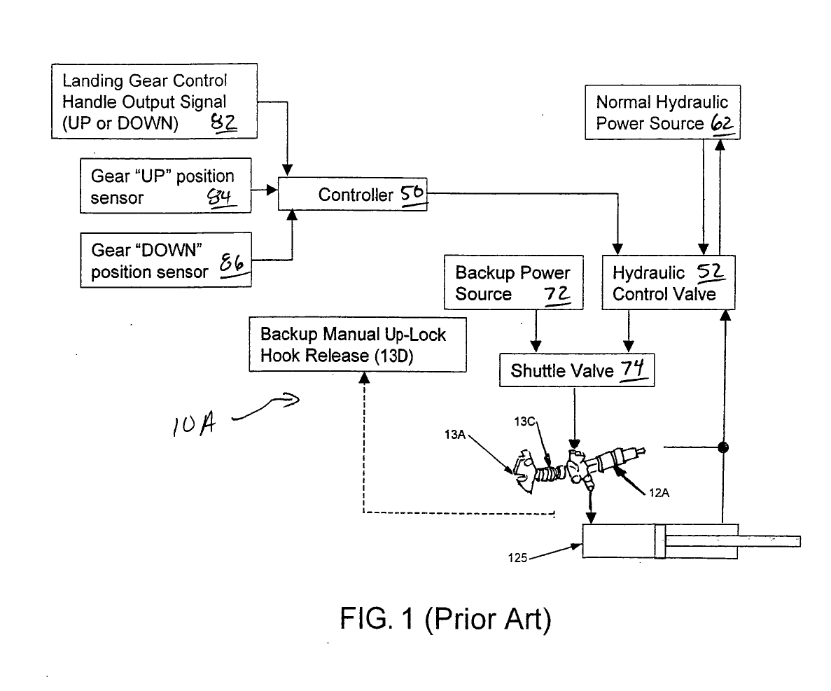

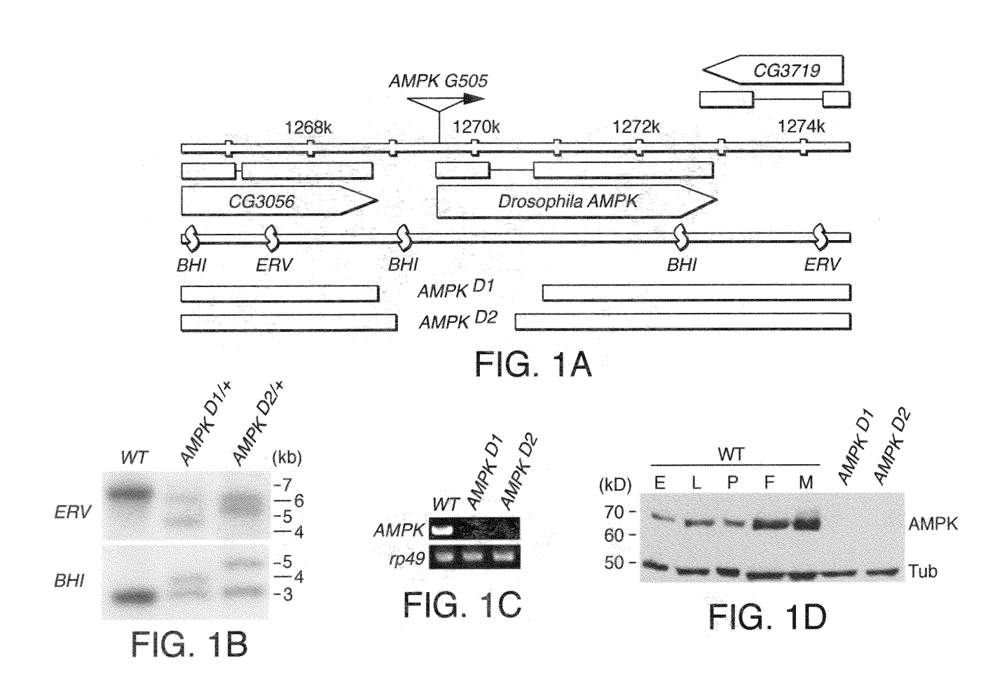

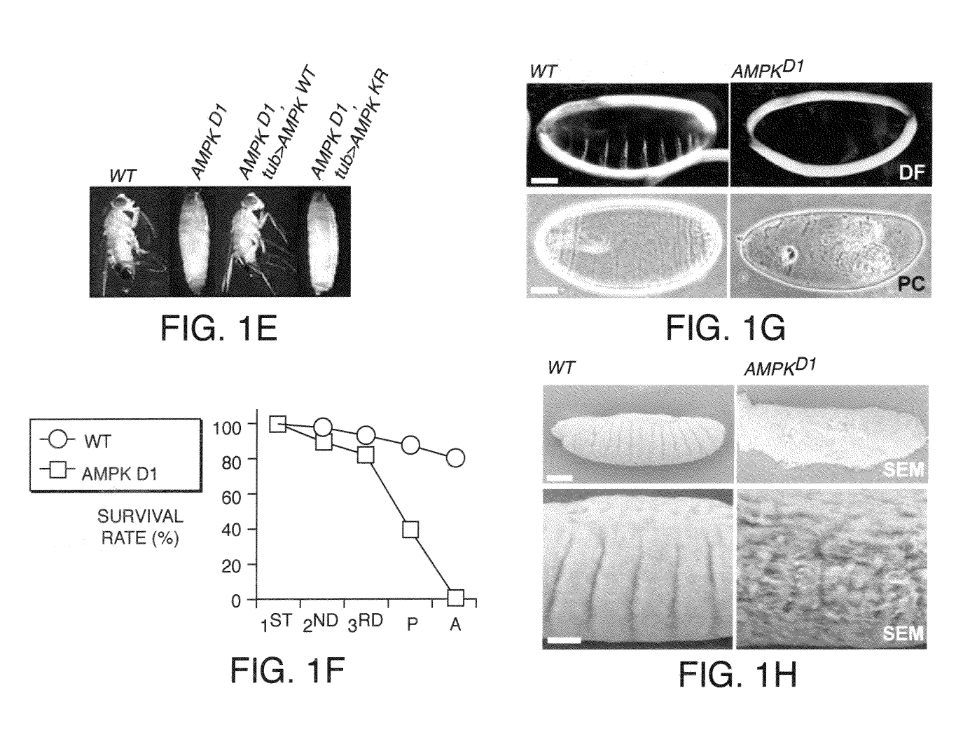

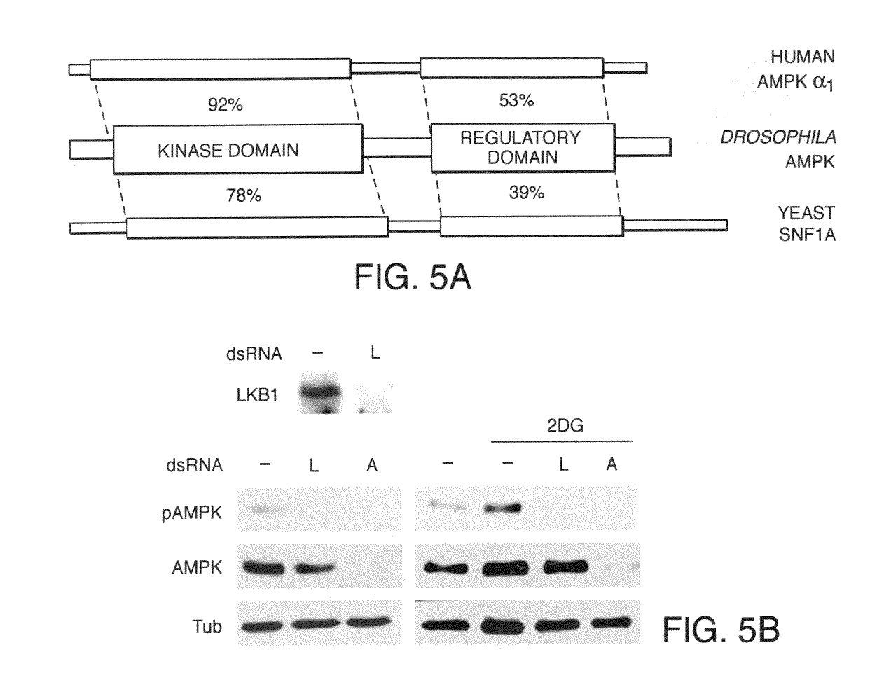

The catalytic subunit of Drosophila AMPK is a single orthologue of its human and yeast counterparts (Kahn et al., Cell Metab. 1, 15-25 (2005); Pan et al., Biochem. J. 367, 179-186 (2002)) (FIG. 5a), and is activated by LKB1 upon energy deprivation (FIG. 5b). By imprecise excision of the EP-element from the AMPK^{G505} line, we generated AMPK-null mutant lines, AMPK^{D1} and AMPK^{D2} (FIG. 1a), whose authenticity was confirmed by Southern blot, RT-PCR, and immunoblot analyses (FIG. 1b-d). Interestingly, all AMPK-null mutant flies were lethal before the mid-pupal stage and failed to enter adulthood (FIG. 1e-f), even in the presence of sufficient nutrients. Although transgenic expression of wild-type AMPK (AMPK^{WT}) allowed AMPK-null mutants to successfully develop into adults (FIG. 1e), the expression of kinase-dead AMPK (AMPK^{KR}) failed to rescue the lethality (FIG. 1e), demonstrating that the phosphotransferase activity of AMPK is crucial for its function. Taken together, we found AMPK to be essential for normal development of Drosophila.

Therefore, we further investigated the developmental role of AMPK by generating AMPK-null germ-line clone (AMPK-GLC) embryos, which are completely deprived of both the maternal and zygotic AMPK proteins. Surprisingly, AMPK-GLC embryos never developed into larvae, showing the requirement of AMPK during embryogenesis. In AMPK-GLC embryos, cuticle structures were severely deformed, and ventral denticle belts were missing (FIG. 1g). Furthermore, the surface of AMPK-GLC embryos was roughened and the columnar structure of the epidermis was disorganized (FIG. 1h), implicating defects in underlying epithelial structures.

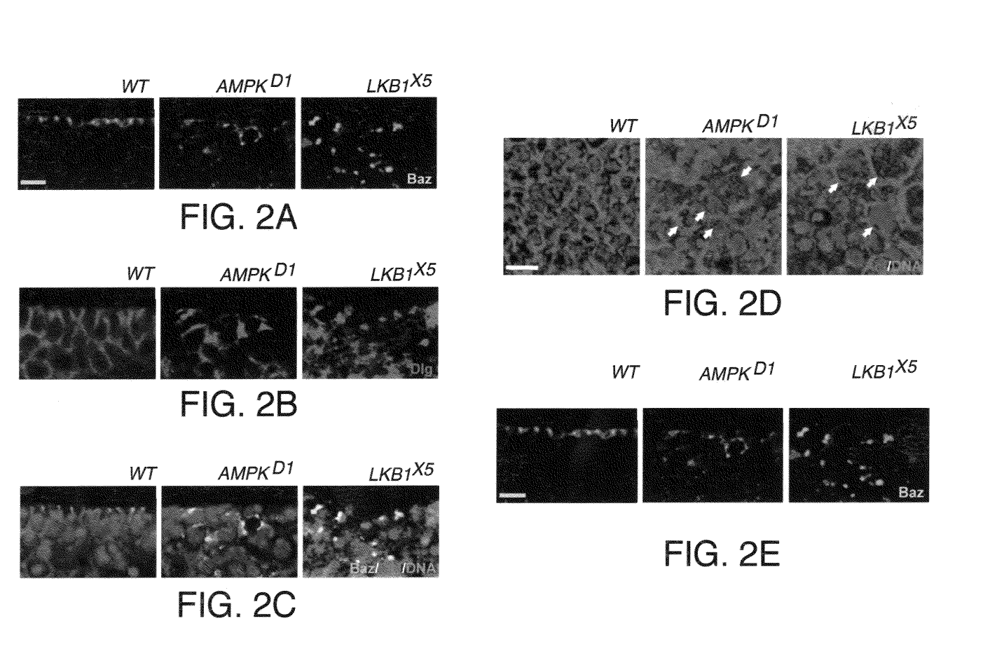

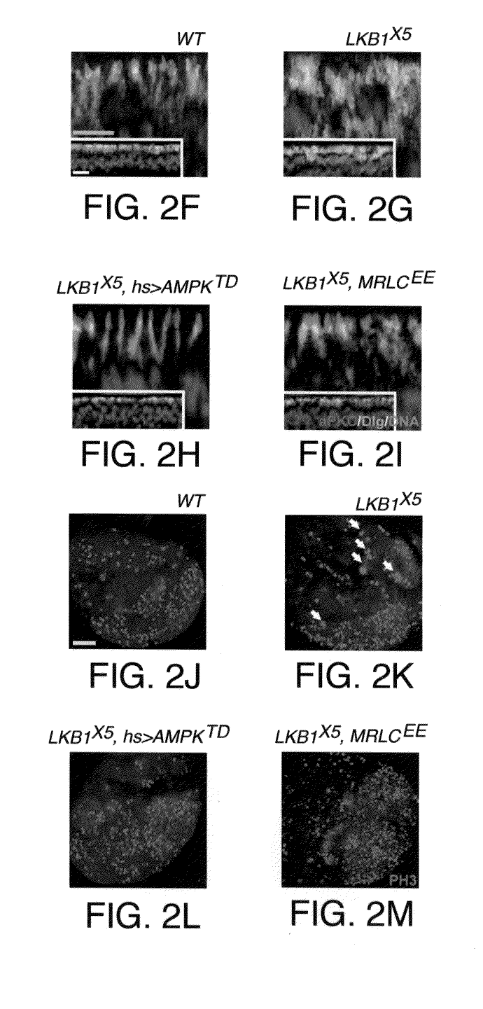

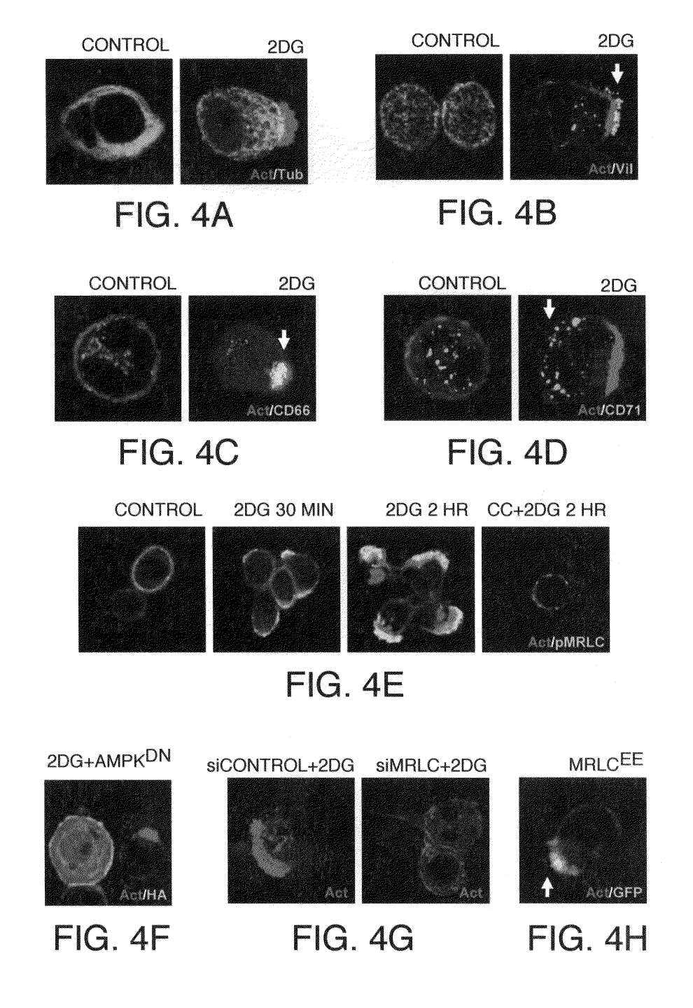

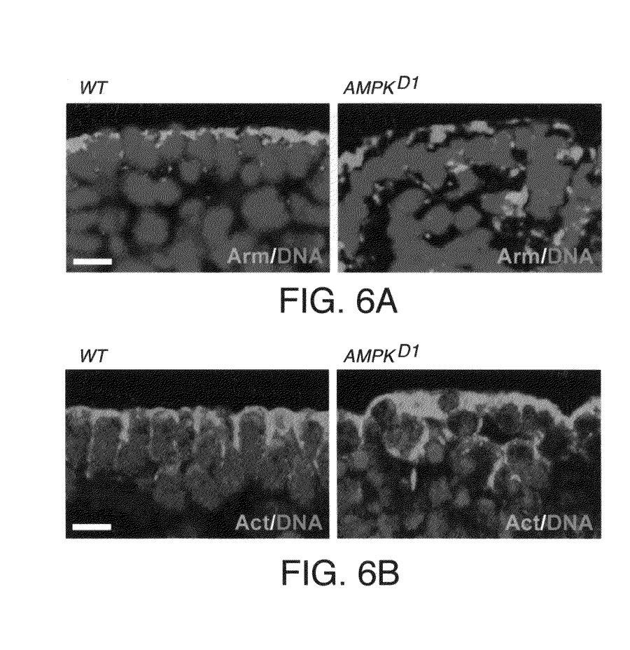

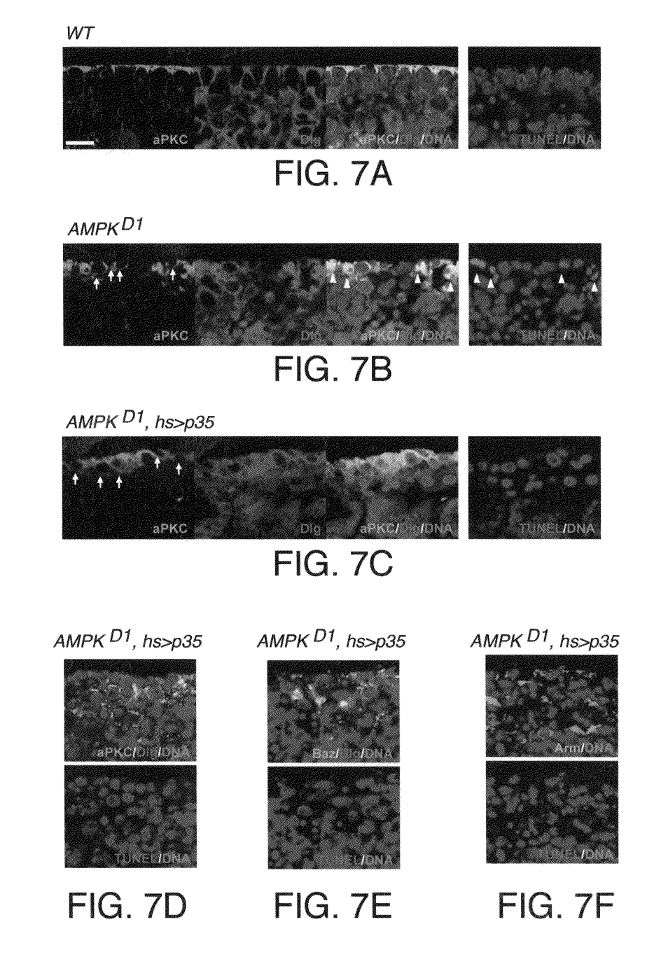

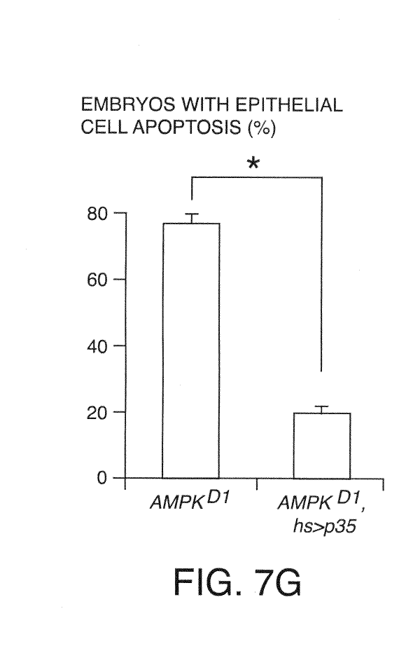

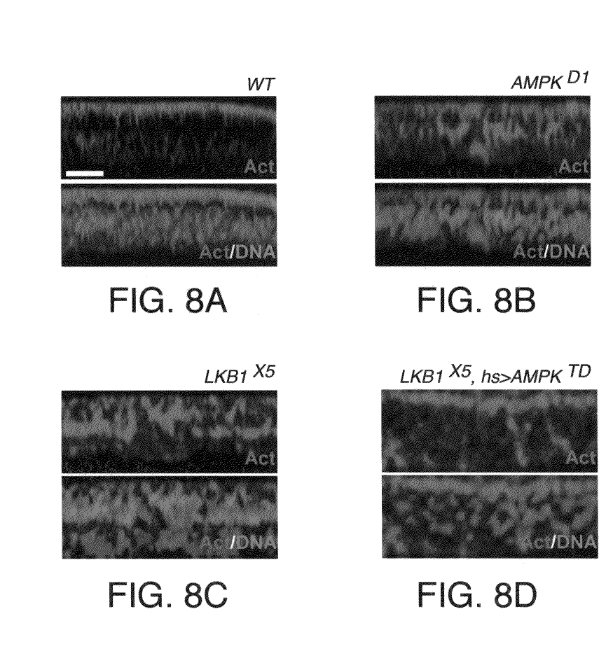

To examine the embryonic epithelial structures, we examined AMPK-GLC epithelia with various polarity markers. Bazooka (Baz, apical complex marker (Knust et al., Science 298, 1955-1959 (2002)) and β-catenin (Arm, adherens junction marker (Knust et al., Science 298, 1955-1959 (2002)) lost their apical localization and were found in various locations around the basolateral cell surfaces (FIGS. 2a, 2c, 6a). The Discs-large (Dlg, basolateral marker (Knust et al., Science 298, 1955-1959 (2002)) was also irregularly distributed throughout the epithelium in AMPK-GLC embryos (FIG. 2 b-c). Moreover, actin staining demonstrated that the AMPK-GLC epithelium contained many unpolarized round cells that had lost contact with the underlying tissue (FIG. 6b). This disorganization of epithelial structures was not a result of cell death, since it could not be restored by overexpression of apoptosis inhibitor p35 (Hay et al., Development 120, 2121-2129 (1994)) (FIG. 7). In addition, wing discs of AMPK-null mutants also displayed defective epithelial organization with ectopic actin structures in the basolateral region (FIG. 8b, compared to FIG. 4a). These results indicate that AMPK is indispensable for epithelial integrity.

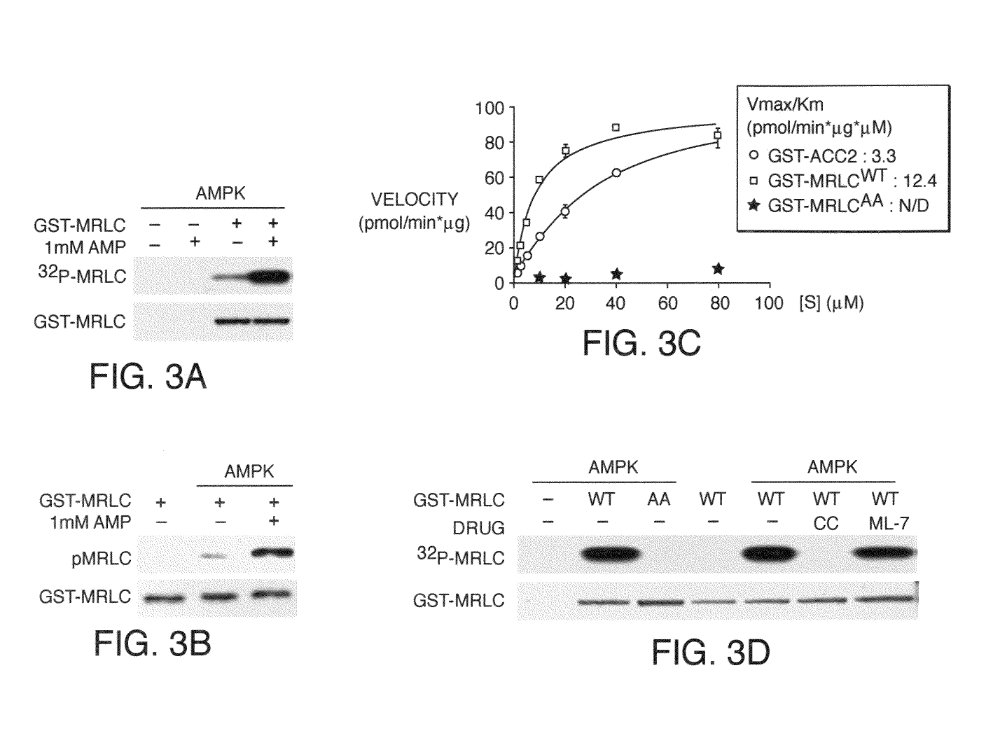

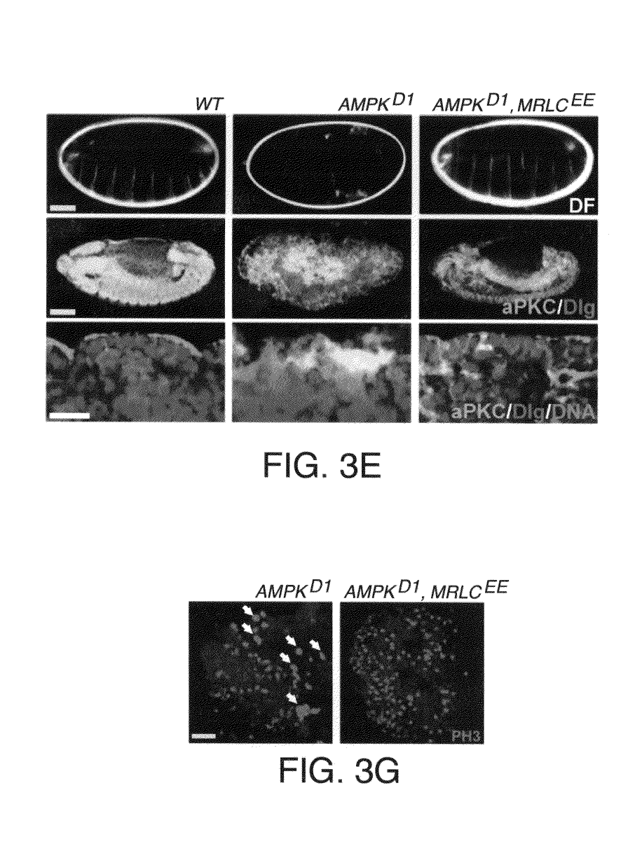

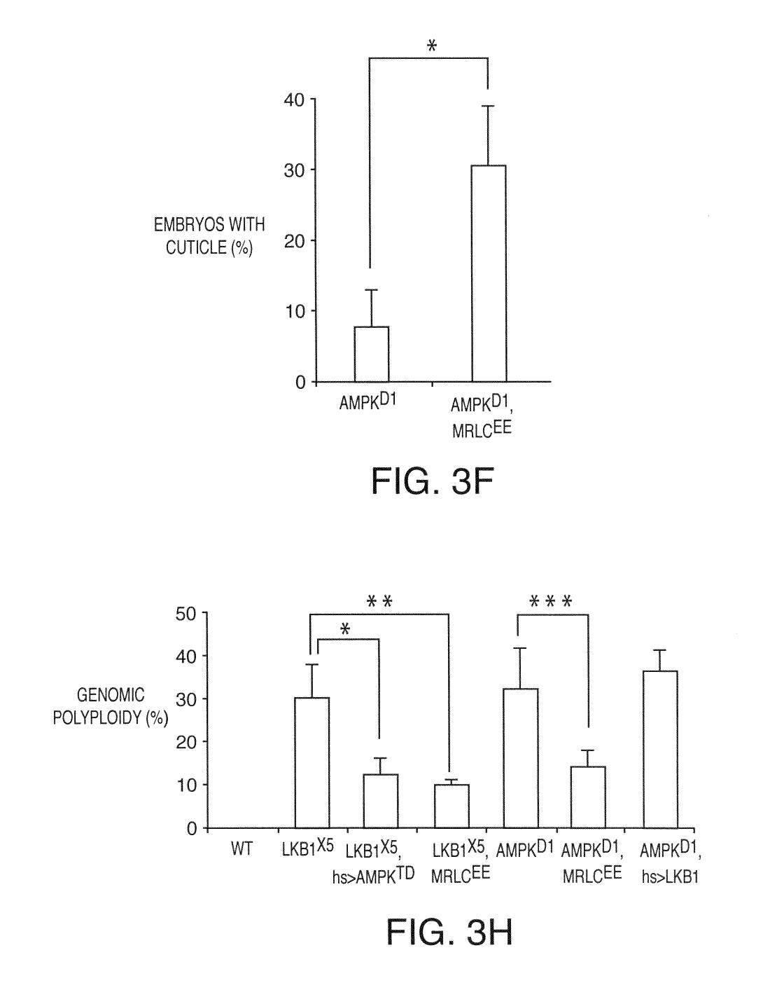

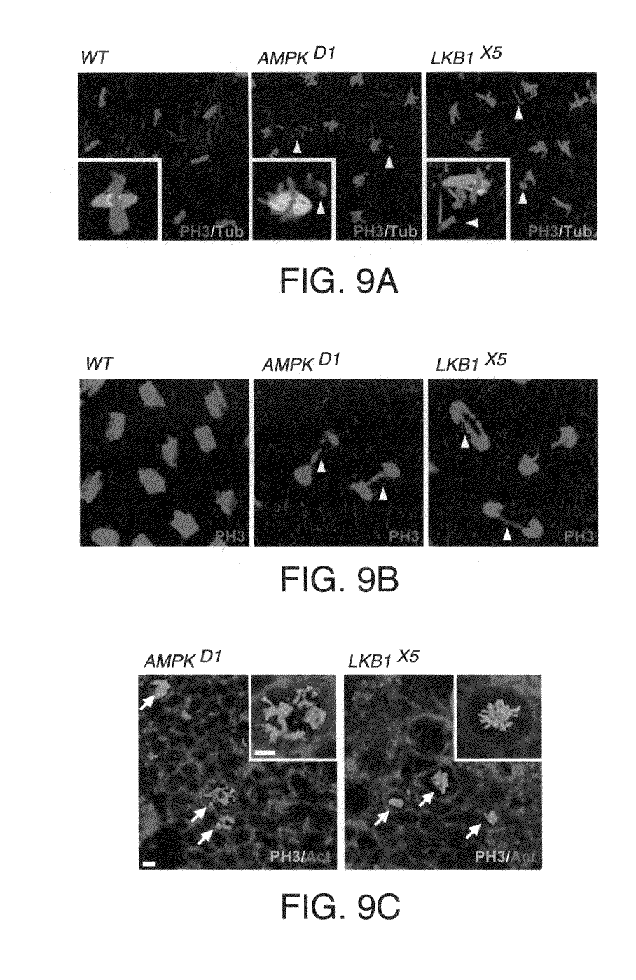

In addition, we found abnormally enlarged nuclei in some cells of AMPK-GLC embryos (FIG. 2d). Mitotic chromosome staining with anti-phospho histone H3 (PH3) antibody demonstrated that AMPK-GLC embryos frequently contained defective mitotic cells with lagging or polyploid chromosomes (FIG. 9). Consistently, aceto-orcein staining of squashed AMPK-null larval brains revealed polyploidy in ˜30% of mitotic cells (FIG. 2e, 3h), and anti-PH3 staining showed a highly increased amount of chromosome content in some of the neuroblasts (FIG. 3g, compared to 2j). These results indicate that AMPK is also required for the maintenance of genomic integrity.

Recently, it has been proposed that LKB1, an upstream kinase of AMPK (Kahn et al., Cell Metab. 1, 15-25 (2005); (Alessi et al., Annu. Rev. Biochem. 75, 137-163 (2006)), is involved in the regulation of epithelial polarity and mitotic cell division (Alessi et al., Annu. Rev. Biochem. 75, 137-163 (2006); (Baas et al., Cell 116, 457-466 (2004); (Martin et al., et al., Nature 421, 379-384 (2003); (Bettencourt-Dias et al., Nature 432, 980-987 (2004)). Indeed, the abnormal polarity and mitosis phenotypes of LKB1-null mutants (FIGS. 2a-e, 2g, 2k, 3h, and 8-11) were highly similar to those of AMPK-null mutants. To test whether AMPK mediates the polarity- and mitosis-controlling functions of LKB1, we expressed constitutively active AMPK (AMPK^{TD}), which is catalytically active even without phosphorylation by LKB1 (data not shown), in LKB1-null mutants. Remarkably, AMPK^{TD} suppressed the epithelial polarity defects (FIGS. 2h, 8d and 11) and the genomic instability (FIGS. 2l and 3h) of LKB1-null mutants, suggesting that AMPK is a critical downstream mediator of LKB1 controlling mitosis and cell polarity.

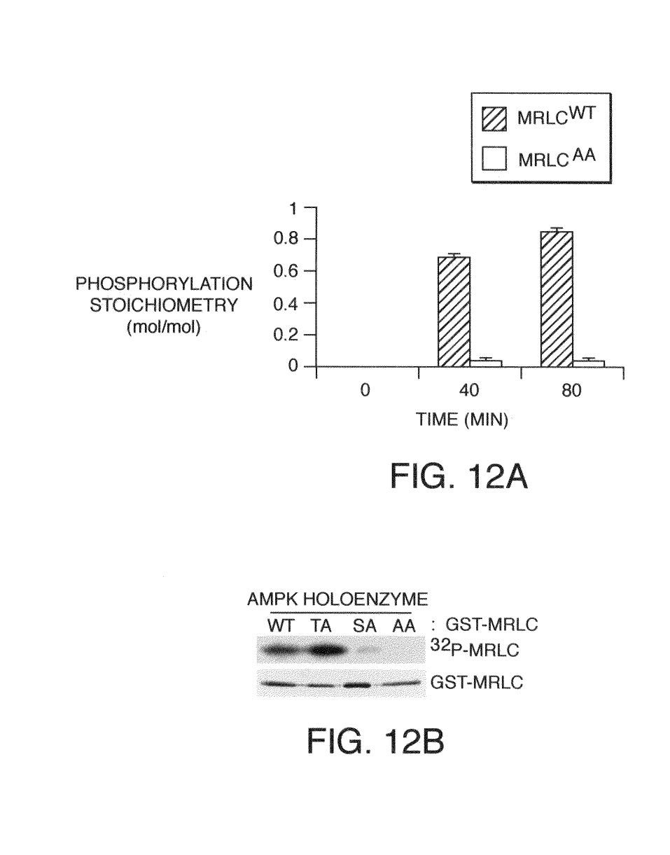

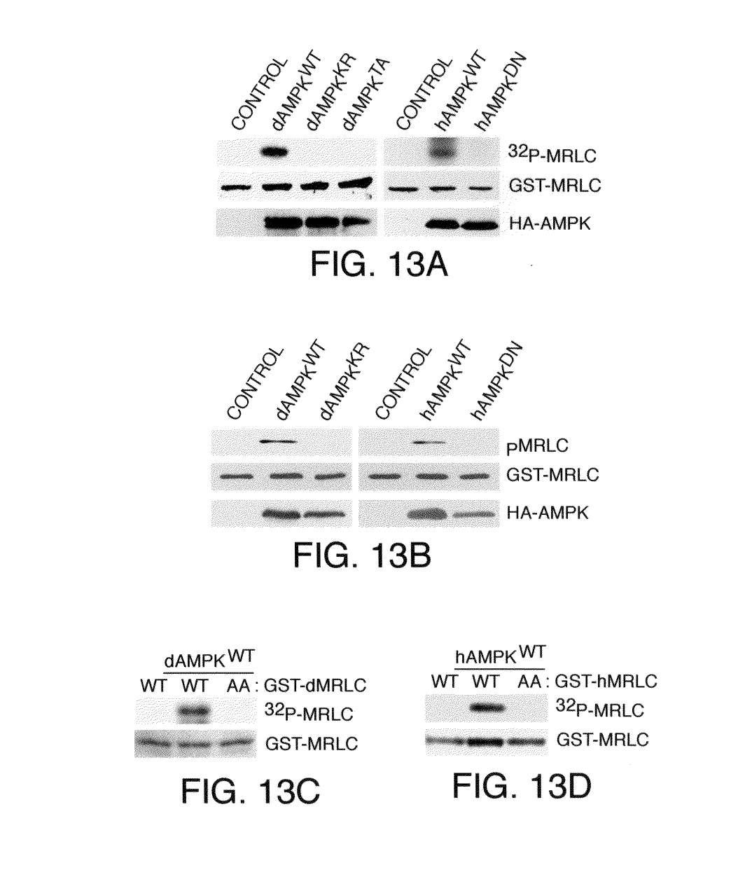

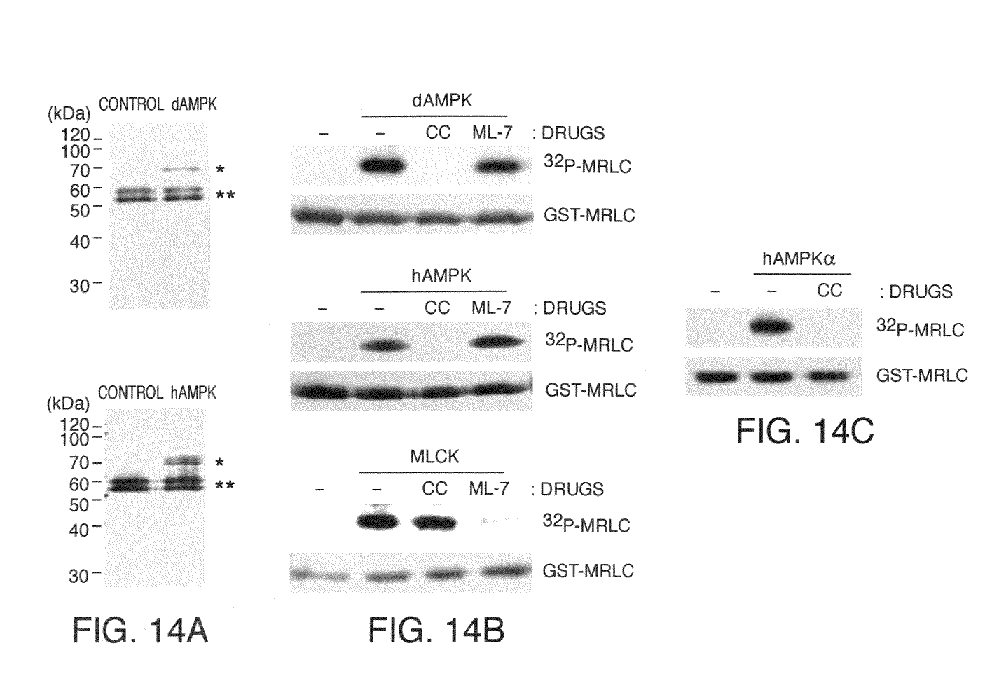

To understand the molecular mechanism underlying the AMPK-dependent control of mitosis and cell polarity, we attempted to identify the downstream targets of AMPK. Intriguingly, MRLC, a critical molecule for the execution of mitosis and cell polarity establishment (Matsumura, Trends Cell Biol. 15, 371-377 (2005); (Jordan et al., J. Cell Biol. 139, 1805-1819 (1997); (Ivanov et al., Mol. Biol. Cell. 16, 2636-2650 (2005); (Edwards et al., Development 122, 1499-1511 (1996)), contains a peptide sequence that can be phosphorylated by AMPK (Michell et al., J. Biol. Chem. 271, 28445-28450 (1996). Therefore, we performed various experiments to evaluate the ability of AMPK to phosphorylate MRLC. AMPK holoenzyme purified from rat liver strongly phosphorylated full-length MRLC, which was further enhanced by the addition of AMP (FIGS. 3 a-b). The phosphorylation of MRLC was more efficient than that of acetyl-CoA carboxylase 2 (ACC2), a representative substrate of AMPK (Kahn et al., Cell Metab. 1, 15-25 (2005) (FIG. 3c), indicating that MRLC is a good in vitro substrate of AMPK. We deduced that this phosphorylation is specifically performed by AMPK since Compound C (CC), a specific inhibitor of AMPK, inhibited the phosphorylation, whereas ML-7, an inhibitor of another MRLC-phosphorylating kinase (MLCK) (Matsumura, Trends Cell Biol. 15, 371-377 (2005), did not (FIG. 3d). A mutant form of MRLC, whose regulatory phosphorylation site (RP site, corresponding to Thr21/Ser22 in Drosophila(Jordan et al., J. Cell Biol. 139, 1805-1819 (1997) and Thr18/Ser19 in human (Matsumura, Trends Cell Biol. 15, 371-377 (2005)) was mutated into non-phosphorylatable alanines, was not phosphorylated by AMPK (FIGS. 3 c-d and FIG. 12), suggesting that MRLC is exclusively phosphorylated at the RP site. Both the human and Drosophila forms of AMPK were able to phosphorylate MRLC from each of the respective species (FIGS. 13, 14), which further demonstrates that the AMPK phosphorylation of MRLC is highly conserved between species.

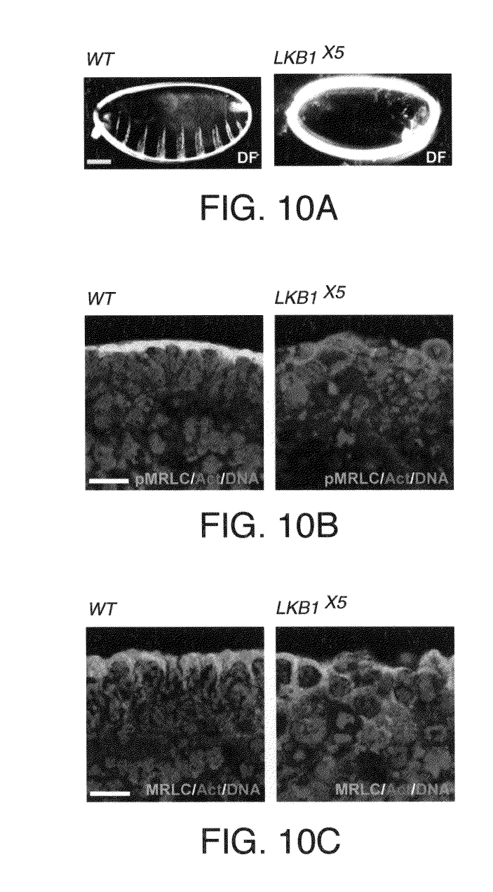

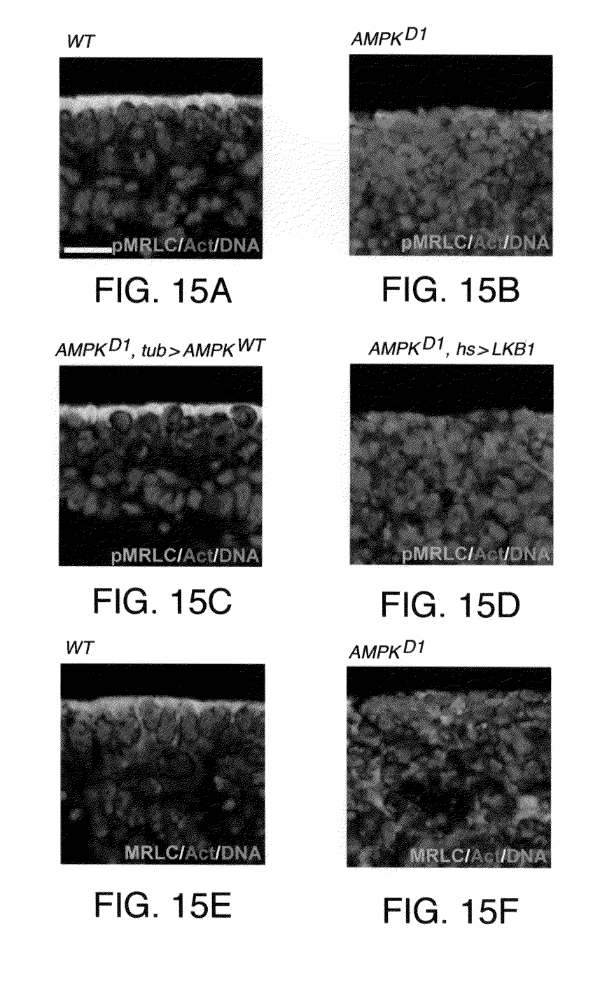

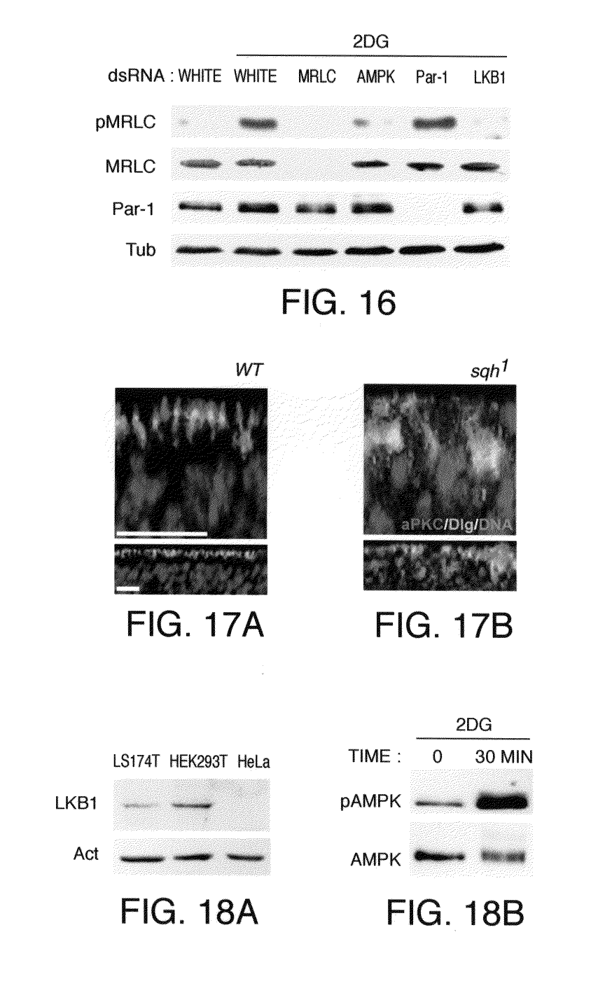

Moreover, we found that MRLC phosphorylation is indeed regulated by AMPK in vivo. The phosphorylation of MRLC was dramatically reduced in AMPK- and LKB1-GLC epithelia when compared to the wild-type epithelia (FIG. 10b, 15 a-b), although the protein level of MRLC was unaffected (FIG. 10c, 15e, 15f). The reduced phosphorylation of MRLC in the AMPK-GLC epithelia was completely restored by transgenic expression of AMPK but not by overexpression of LKB1 (FIG. 15c-d). Furthermore, in Drosophila S2 cells, energy deprivation induced by 2-deoxyglucose (2DG) enhanced MRLC phosphorylation, which was suppressed by dsRNA-mediated silencing of LKB1 or AMPK (FIG. 16). Collectively, these data strongly suggest that MRLC is specifically phosphorylated by AMPK both in vitro and in vivo.

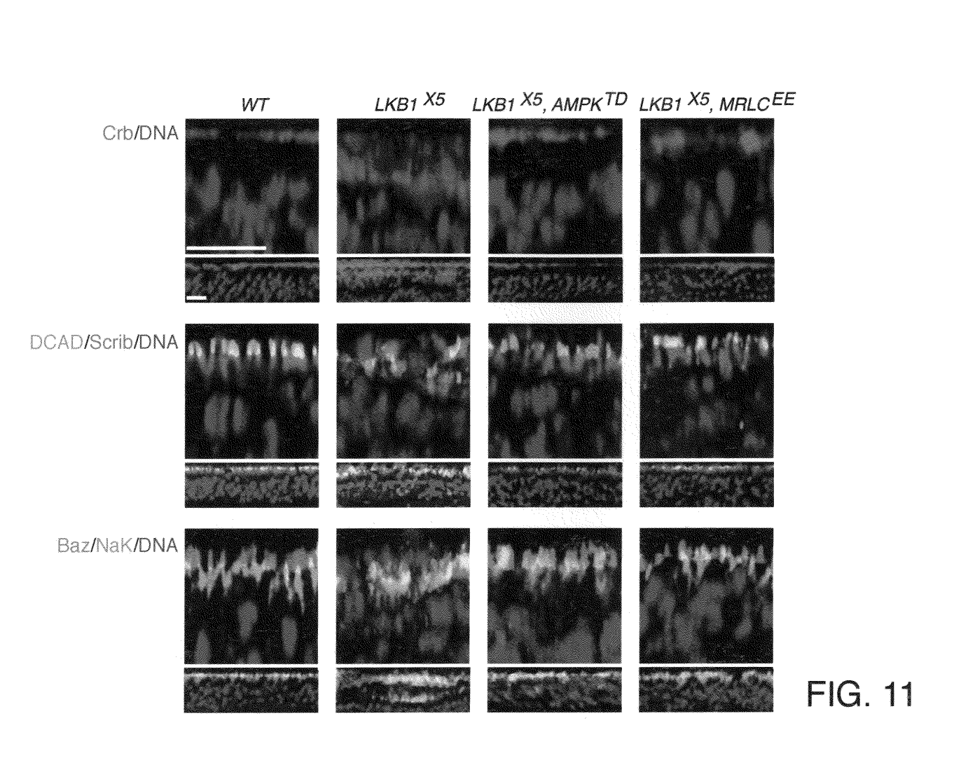

To find out whether the phosphorylation of MRLC is critical for the physiological functions of AMPK, we expressed an active form of MRLC (Jordan et al., J. Cell Biol. 139, 1805-1819 (1997) (MRLC^{EE}), whose RP site was mutated into phosphomimetic glutamates, in AMPK-GLC embryos. Strikingly, MRLC^{EE} rescued the epithelial polarity defects caused by the loss of AMPK (FIG. 3e), and increased the percentage of cuticle-forming embryos from ˜10% to ˜30% (FIG. 3f). MRLC^{EE} also restored the epithelial polarity defects of LKB1-null wing imaginal discs (FIG. 2i and FIG. 11). Furthermore, the genomic polyploidy of AMPK- and LKB1-null larval brain neuroblasts was suppressed by the expression of MRLC^{EE} (FIGS. 2m, 3g, 3h). Therefore, we conclude that MRLC is a critical downstream target of AMPK controlling cell polarity and mitosis.

Notably, the larval brains of MRLC loss-of-function mutants (spaghetti-squash (Kahn et al., Cell Metab. 1, 15-25 (2005)) displayed extensive polyploidy (˜40% of mitotic neuroblasts) (Karess et al., Cell 65, 1177-1189 (1991), and their imaginal discs showed severe disorganization in epithelial structure (FIG. 17), similar to those of LKB1- and AMPK-null mutants. These phenotypic similarities further support our conclusion that MRLC is an important functional mediator of LKB1 and AMPK.

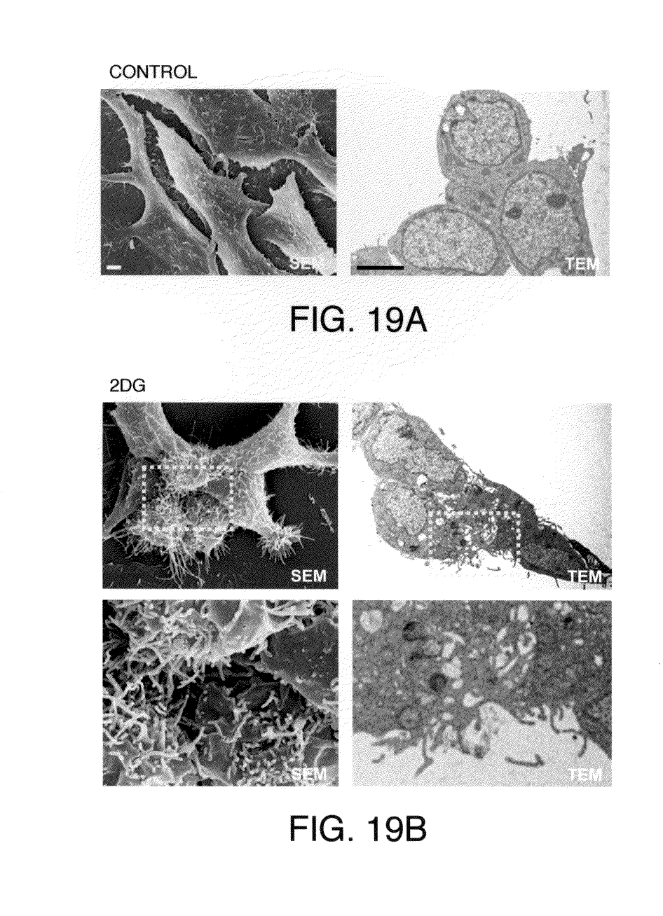

Finally, we questioned whether AMPK is critical for directing cell polarity in mammalian cells as well. To assess this, we examined whether the activation of AMPK by 2DG treatment (FIG. 18) could induce polarization of unpolarized epithelial cells such as LS174T, which can be polarized by the activation of LKB1 (Baas et al., Cell 116, 457-466 (2004)), in a cell-autonomous manner. Surprisingly, upon 2DG treatment, LS174T cells underwent a dramatic change in cell shape to have polarized actin cytoskeleton with a brush border-like structure (FIGS. 4a, 19, 20a, 20f). Moreover, while brush border marker villin (Baas et al., Cell 116, 457-466 (2004)) (FIG. 4b), apical marker CD66/CEA (Baas et al., Cell 116, 457-466 (2004)) (FIG. 4c), and basal marker CD71/transferrin (Baas et al., Cell 116, 457-466 (2004)) (FIG. 4d) were distributed throughout untreated cells, they became dramatically polarized upon 2DG treatment (FIG. 4b-d), supporting that the activation of AMPK by energy deprivation is sufficient to induce complete polarization of LS174T cells.

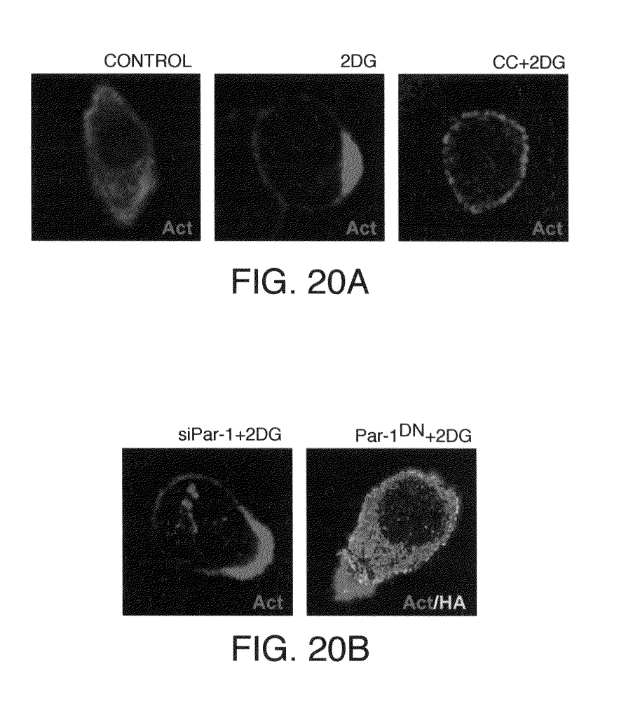

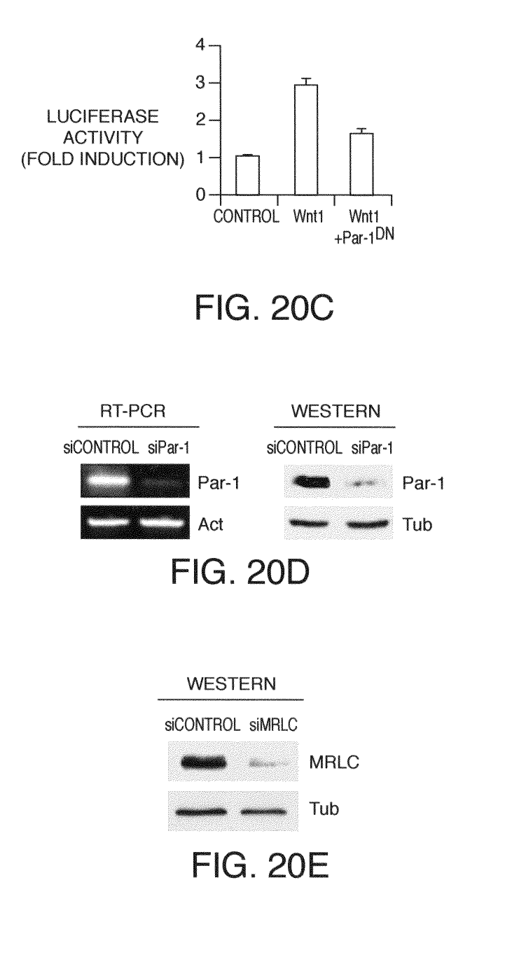

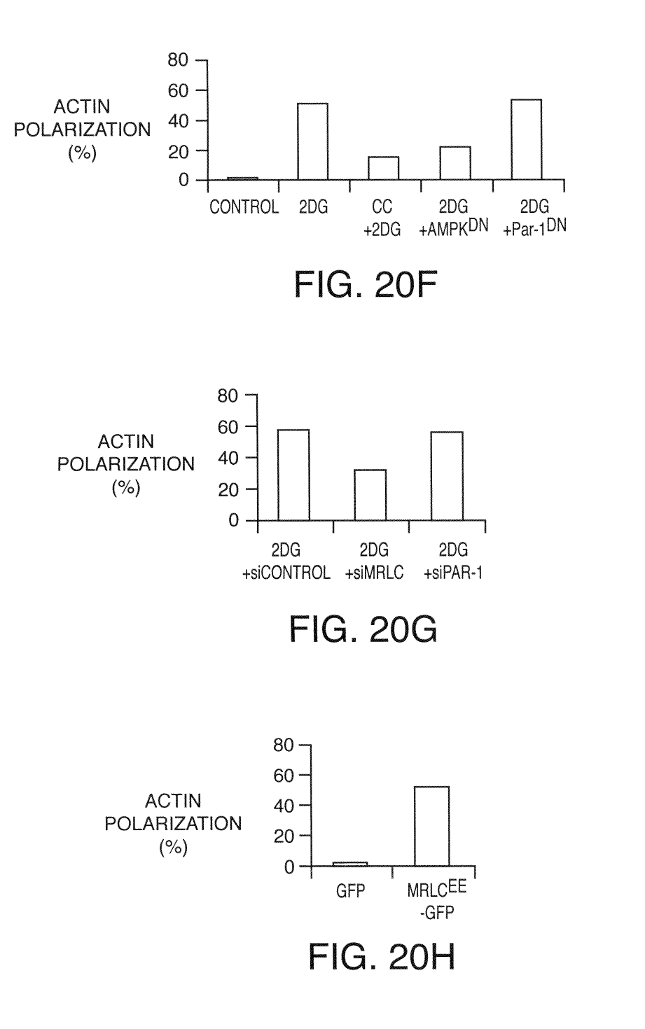

We also found that the phosphorylation of MRLC by AMPK is involved in the energy-dependent polarization of LS174T cells. Phosphorylated MRLC was colocalized with the 2DG-induced polarized actin structures (FIG. 4e), and this phosphorylation, as well as the actin polarization, was suppressed by the AMPK-specific inhibitor CC (FIGS. 4e, 20a, 20f). Overexpression of dominant-negative AMPK (AMPK^{DN}) and siRNA-mediated inhibition of MRLC (siMRLC) also blocked the polarization (FIG. 4 f-g, 20 e-g), although inhibition of Par-1, another downstream kinase of LKB1 (Alessi et al., Annu. Rev. Biochem. 75, 137-163 (2006)), by Par-1 siRNA (siPar-1) or overexpression of dominant-negative Par-1 (Par-1^{DN}) failed to suppress it (FIG. 20 b-d, f, g). More strikingly, human MRLC^{EE} itself was sufficient to polarize LS174T cells, even without energy deprivation (FIGS. 4h, 20h), showing that phosphorylation of MRLC is critical for the AMPK-dependent polarization.

Until now, the importance of AMPK has been limited to its role as a regulator of metabolism (Kahn et al., Cell Metab. 1, 15-25 (2005)). However, by generating the first animal model with no AMPK activity, we arrived at characterizing a novel function of AMPK: AMPK regulates mitotic cell division and epithelial polarity at the downstream of LKB1, by controlling the activity of MRLC through direct phosphorylation. The present invention, in some embodiments, employs this finding for animals models, drug screens, and related diagnostic and therapeutic methods and compositions. Our findings revealed a novel link between energy status and cell structures, providing a new perspective to the diverse molecular function of AMPK. These finding regarding the cell structure-controlling function of AMPK with respect to the various metabolic and physiological contexts will allow a better understanding of AMPK-related diseases such as cancer and diabetes (Kahn et al., Cell Metab. 1, 15-25 (2005); (Alessi et al., Annu. Rev. Biochem. 75, 137-163 (2006); (Luo et al., Trends Pharmacol. Sci. 26, 69-76 (2005)).

### EXEMPLARY USES AND EMBODIMENTS OF THE PRESENT INVENTION

The energy sensing enzyme AMP-activated protein kinase (AMPK) was, prior to the present invention, primarily considered as a controller of metabolic responses that were related to diabetes and exercise physiology. The present invention provides the first animal model with no AMPK activity. This animal model successfully uncovered novel physiological functions of AMPK, indicating certain applications in disease treatment and drug development.

1. AMPK-Null Animal Models

Although many studies have investigated the biochemical functions of AMPK for decades, the in vivo function of AMPK has been hard to elucidate, due to genetic redundancy of the AMPK gene in most animal systems. Using Drosophila, which has only a single orthologue of AMPK, the present invention provides the first AMPK-null model animal. The animal models of the present invention can be used, for example, for drug screening and to elucidate biochemical pathways (e.g., in humans). The animal models of the present invention enabled us effective investigation into physiological functions of AMPK and identification of its novel downstream mediator MRLC. The animal models of the present invention may be used, for example, in identification of new drug target molecules or drug candidates for AMPK-related diseases, such as diabetes and cancer.

2. AMPK as a Target of Cancer Treatment

AMPK-null mutants demonstrated severe defects in epithelial polarity and genomic stability, highly similar to those caused by the loss of its upstream kinase LKB1, a tumor suppressor mutated in a wide range of sporadic tumors and Peutz-Jegher's syndrome. Constitutively active AMPK restored the LKB1-null phenotypes, showing that AMPK is a critical mediator of LKB1 in maintaining epithelial integrity and genomic stability. Because loss of cell polarity and genomic instability are ultimately correlated with more aggressive and invasive cancer growth, the data in the present application indicates that AMPK as an important target of cancer treatment and prevention. As such, in certain embodiments, the present invention provides for the use of activators of AMPK (e.g., meformin) to treat cancer (e.g., by promoting polarization of cancer cells, and consequently inhibit their invasive characteristics). Support for such treatment is the finding that activation of AMPK induced complete polarization in a colon cancer cell LS174T.

3. Roles of AMPK in the Intestine

In addition to inducing complete polarization of epithelial cells, activation of AMPK by energy depletion in LS174T also induced a brush-border like structure made up of dense patch of microvilli, where absorption of nutrients takes place. This data indicates that AMPK enhances nutrient absorption in intestines under starvation condition. Moreover, a gastrointestinal pathogen S. typhimurium disrupts epithelial cell polarity to disassemble the intestinal barrier, indicating that activation of AMPK can promote absorption of nutrient and prevent pathogen invasion in intestinal system.

In other embodiments, compounds are administered (e.g., to the intestines of a patient) to prevent the activation of AMPK, and thereby inhibit absorption of nutrients by the intestine (e.g., to treat obesity or for weight loss).

4. Role of AMPK in the Kidney

The polarity of the kidney epithelial cells appears to be regulated by AMPK. Many diseases-related to the polarity may be controlled by the inhibitors and activators of AMPK. As such, in certain embodiments, a patient with kidney type disease is administered either an AMPK activator or an AMPK inhibitor.

5. Drugs Targeting AMPK

5-1. Activators for AMPK

As described above, AMPK is a key target molecule to treat metabolic syndromes such as diabetes. In fact, an AMPK-activating agent, metformin has been used as anti-type 2 diabetes drugs for more than 50 years (the most popular anti-diabetic drug in the United States and one of the most prescribed drugs overall, with nearly 35 million prescriptions filled in 2006 for generic metformin alone). So, these drugs can be used to treat cancer or promote intestinal absorption without further clinical trials. Therefore, in certain embodiments, a patient is administered an AMPK activator (e.g., metformin or derivative thereof) to treat a disease (e.g., cancer).

5-2. Other Known Activators for AMPK

Other known activators of AMPK that could be used with the methods and compositions of the present invention include, but are not limited to: AICAR, Resveratrol, and Thiazolidinedione. AICAR (5-aminoimidazole-4-carboxamide-1-β-D-ribofuranoside)—in the cell AICAR is converted to ZMP, an AMP analog that has been shown to activate AMPK. Resveratrol—increases the activity of SIRT1 and animal life span, and it also increases AMPK activity by SIRT1 independent mechanism. Thiazolidinedione (TZD)—a PPAR gamma activator, which activates AMPK in PPAR gamma-independent manner.

5-3. Inhibitors for AMPK

One example of an AMPK inhibitor is Compound C: A cell-permeable pyrrazolopyrimidine compound that acts as a potent, selective, reversible, and ATP-competitive inhibitor of AMPK.

### Methods Summary

Fly strains. AMPK^{D1} and AMPKD2 lines were generated by imprecise excision (Lee, et al., FEBS Lett. 550, 5-10 (2003)) of AMPK^{G505} line (GenExel Inc.). UAS constructs were microinjected into w¹¹¹⁸ embryos. LKB1- and MRLC-mutant lines were described previously (Jordan et al., J. Cell Biol. 139, 1805-1819 (1997); (Karess et al., Cell 65, 1177-1189 (1991); (Lee et al., Cell Death Differ. 13, 110-1122 (2006)). Other lines were obtained from the Bloomington Stock Center.
Microscopic analyses of Drosophila tissues. Dark field, phase contrast, and SEM analyses were performed to visualize embryonic cuticle structure. Immunostaining with various antibodies were performed to visualize embryonic epithelial structure. Brain squash preparation was used to analyze genomic integrity.
Kinase assays. Phosphotransferase activities were determined in a reaction mixture consisting of HEPES-Brij Buffer, ATP (γ-³²P), and protein substrates. Protein levels were visualized by immunoblot or Coomassie staining. Phosphorylated proteins were visualized by phospho-specific immunoblot or ³²P-autoradiography. Incorporated phosphates were quantified using P81 filter binding assay.
Drosophila strains. The AMPK^{G505} fly line with an EP-element inserted at the AMPK locus was obtained from the Genisys Collection of EP lines (GenExel Inc., Korea), and subjected to standard imprecise excision (Lee, et al., FEBS Lett. 550, 5-10 (2003); (Lee et al., Cell Death Differ. 13, 110-1122 (2006)). HA-tagged AMPK (AMPK^{WT}) and its mutant forms (AMPK^{KR}, AMPK^{TA} and AMPK^{TD}) were subcloned into the pUAST vector and microinjected into w¹¹¹⁸ embryos. LKB1^{X5}, UAS-LKB1, spaghetti-squash¹ (sqh¹), and MRLC^{EE} (also referred to as sqh^{EE}) lines were described previously (Jordan et al., J. Cell Biol. 139, 1805-1819 (1997); (Karess et al., Cell 65, 1177-1189 (1991); (Lee et al., Cell Death Differ. 13, 110-1122 (2006)). The Gal4 lines, balancer lines, UAS-p35 line, FRT/FLP lines, and dominant female-sterile lines were obtained from the Bloomington Stock Center.
Antibodies. His-tagged full-length Drosophila AMPK protein was purified by nickel column and injected into rabbits to generate anti-Drosophila AMPK antibody. In addition, anti-Drosophila LKB1 (Lee et al., Cell Death Differ. 13, 110-1122 (2006), anti-phospho-Ser19 human MRLC (Cell Signaling, #3671 was used to detect phospho-Ser22 Drosophila MRLC (Lee et al., Mol. Biol. Cell 15, 3285-3295 (2004)), and #3675 was used to detect phospho-Ser19 human MRLC (Sakurada et al., Am. J. Physiol. 274, 1563-1572 (1998)), anti-full-length human MRLC (FL-172, Santa Cruz, this antibody also recognizes Drosophila MRLC), anti-phospho-Thr172 human AMPK (40H9, Cell Signaling, this antibody also recognizes phospho-Thr184 Drosophila AMPK (Pan et al., Biochem. J. 367, 179-186 (2002)), anti-human AMPK (Cell Signaling), anti-human LKB1 (Upstate), anti-villin (CWWB1, Abcam), anti-CD66 (1C3, Abcam), FITC-conjugated anti-CD71 (L01.1, BD Biosciences), anti-HA [3F10 (Roche) was used for immunoblot, 12CA5 (Roche) was used for immunoprecipitation, and 6E2 (Cell Signaling) was used for immunocytochemistry.], anti-atypical PKC (Santa Cruz), anti-phospho-Ser10 histone H3 (Upstate), anti-Bazooka (a gift from Dr. A. Wodarz), anti-Drosophila Par-1 (a gift from Dr. D. St Johnston), anti-GST (Upstate), anti-human Par-1 (183.A10.A3, Upstate), anti-Scribble (a gift from Dr. C. Q. Doe), anti-Armadillo (N2 7A1, DSHB), anti-Discs large (4F3, DSHB), anti-Crumbs (Cq5, DSHB), anti-Drosophila E-cadherin (DCAD2, DSHB), anti-Na⁺/K⁺ ATPase (a5, DSHB), anti-β-tubulin (E7, DSHB), and anti-actin (JLA20, DSHB) antibodies were used for immunostaining and immunoblot analyses. TRITC-labeled phalloidin (Sigma) and Hoechst 33258 (Sigma) were also used to visualize filamentous actins and DNA, respectively.
Confirmation of AMPK-null mutants. Deletion sites of AMPK^{D1} and AMPK^{D2} were determined by genomic-PCR analyses. EP-element insertion in AMPK^{G505} mutant (triangle) and genomic deletions in AMPK^{D1} (1,268,785-1,270,743th basepair) and AMPK^{D2} (1,269,080-1,270,246th basepair) mutants are indicated in FIG. 1a. Nucleotide numbering in FIG. 1a was done according to the Drosophila melanogaster chromosome X sequence (release v5.1). For Southern blot analyses (Lee, et al., FEBS Lett. 550, 5-10 (2003); (Park et al., Nature 441, 1157-1161 (2006)), 10 μg of total genomic DNA from w¹¹¹⁸, AMPK^{D1}/+, AMPK^{D2}/+ flies was digested with EcoRV or BamHI (KOSCHEM, Korea). Drosophila AMPK full-length ORF was used as a probe. For RT-PCR analyses (Lee, et al., FEBS Lett. 550, 5-10 (2003); (Lee et al., Cell Death Differ. 13, 110-1122 (2006); (Park et al., Nature 441, 1157-1161 (2006)), total RNA from the third instar larvae was extracted with Easy-Blue™ (Intron, Korea), and reversely transcribed using Maxime RT premix kit (Intron, Korea). Then, the following primers were used to amplify the AMPK transcripts by PCR: 5′-GATCACACGCGTCAAGGTGGCC-3′ and 5′-GGTCTCGATGCACGAT CATGTGCC-3′. rp49 transcripts were amplified as previously described (Lee, et al., FEBS Lett. 550, 5-10 (2003); (Park et al., Nature 441, 1157-1161 (2006)).
Microscopic examination of Drosophila embryos. Dark field (DF) and phase contrast (PC) analyses were performed using a light microscope (DM-R, Leica). Embryonic cuticles were prepared as previously described (Lee et al., Cell Death Differ. 13, 110-1122 (2006). To quantify the number of embryos with cuticle for FIG. 3f, we performed three replicate experiments (60-90 embryos were observed for each experiment) to calculate the proportion of the number of embryos with ventral denticle belts to the total number of embryos. The average ratio of the three experiments is presented in % as a bar graph, and the standard deviation is indicated as error bars. A P-value was calculated using one-way ANOVA analysis. For scanning electron microscopy (SEM) analyses, embryos were dechorinated with 50% bleach, fixed with a mixture of 4% paraformaldehyde solution and heptane, then devitellinized with a methanol-heptane mixture. Rehydrated embryos were dried in air for 3 min and frozen for SEM analyses. SEM images were obtained by LEO 1455VP Electron Microscopy System in the VPSE (Variable Pressure Secondary Electron) mode.
Preparation of Mitotic Figures in Larval Brain. Larval Brains were Dissected in isotonic saline. The brains were first incubated for 90 min in 5×10⁻⁵ M colchicine in saline, then were hypotonically shocked for 10 min in 0.5% sodium citrate. Finally, the brains were fixed and stained as described previously (Karess et al., Cell 65, 1177-1189 (1991)). Cytological examination was performed under a light microscope (DM-R, Leica). For quantification of genomic polyploidy in FIG. 3h, we calculated the proportion of the number of polyploidy mitotic neuroblasts to the total number of mitotic neuroblasts. We examined 400-700 mitotic cells from more than three different brains of the same genotype. The average of the ratios among brains is presented in % as bar graphs, and the standard deviation is indicated as error bars. P-values were calculated using one-way ANOVA analyses.
In vitro kinase assay. Protein kinase assay was performed in a solution consisting of HEPES-Brij Buffer, 0.2 mM ATP (with 0.5 μCi/μl γ-³²P-ATP for radioactive assay), and 1 μg or indicated amount of protein substrate at 30° C. for 20 min. For FIG. 3d and FIG. 14 b-c, 10 μM Compound C or 10 μM ML-7 were added to the reaction mixture as indicated. For FIGS. 3 a-b, 0.3 mM AMP was added to the mixture as indicated. Except for FIGS. 3 a-b, all reaction mixtures contained 0.3 mM AMP. Except for FIG. 3c and FIG. 12a, the assay samples were subjected to SDS-PAGE, and then autoradiography or immunoblotting was performed. For FIGS. 3c and 12a, the radioactive assay samples were subjected to P81 filter binding assay (Monfar et al., Mol. Cell. Biol. 15, 326-337 (1995). Vmax and Km values were obtained by nonlinear regression analysis (curve fitting) using GraphPad Prism version 4.0 (Graphpad Software). Vmax values (+standard errors) for GST-ACC2 and GST-MRLC^{WT} were 113.9±3.216 μmol/μg*min and 98.93±5.264 μmol/μg*min, respectively. Km values (+standard errors) of GST-ACC2 and GST-MRLC were 34.37±2.114 μM and 8.001±1.417 μM, respectively. Because kinetic parameters for GST-MRLC^{AA} did not converge, curve fitting was not possible.

Detailed methods on molecular biology, fly genetics, immunoblot, immunostaining, preparation of kinases and their substrates, Drosophila S2 cell culture, and mammalian cell culture are described below.

Drosophila genetics. The germ-line clone (GLC) embryos were generated using the autosomal FLP-DFS technique (Chou et al., Genetics 144, 1673-1679 (1996)). In detail, to generate AMPK-GLC embryos, AMPK^{D1} FRT14A/FM6 females were crossed with ovo^{D1} FRT14A/Y; hs-FLP males. Among the progenies, ovo^{D1} FRT14A/AMPK^{D1} FRT14A; hs-FLP/+ females were selected and crossed with FM7Act-GFP/Y males. Only GFP-negative embryos were collected from this cross to obtain AMPK-GLC embryos that are deprived of both the maternal and zygotic AMPK protein. To produce wild-type germ-line clone embryos, ovo^{D1} FRT14A/FRT14A; hs-FLP/+ females were crossed with FM7Act-GFP/Y males. To produce AMPK-GLC embryos expressing AMPK^{WT}, ovo^{D1} FRT14A/AMPK^{D1} FRT14A; hs-FLP/+; tub-Gal4 UAS-AMPK^{WT}/+ females were crossed with FM7 Act-GFP/Y males. To produce AMPK-GLC embryos expressing the p35 protein, ovo^{D1} FRT14A/AMPK^{D1} FRT14A; hs-FLP/+ females were crossed with FM7 Act-GFP/Y; hs-Gal4 UAS-p35/CyO Act-GFP males. To produce AMPK-GLC embryos expressing LKB1 protein, ovo^{D1} FRT14A/AMPK^{D1} FRT14A; hs-FLP/+ females were crossed with FM7Act-GFP/Y; hs-Gal4 UAS-LKB1/CyO Act-GFP males. For expression of p35 or LKB1 in AMPK-GLC embryo, eggs were collected and aged at 30° C. To produce AMPK-GLC embryos containing MRLC^{EE} protein, ovo^{D1} FRT14A/AMPK^{D1} FRT14A; hs-FLP/+; MRLC^{EE}/+ females were crossed with FM7Act-GFP/Y males. To generate LKB1-GLC embryos, y w hs-FLP; FRT82B LKB1^{X5}/TM6B females were crossed with FRT82B ovo^{D1}/TM3 Sb males. Among the progenies, y w hs-FLP/+; FRT82B LKB1^{X5}/FRT82B ovo^{D1} females were selected to obtain LKB1-GLC embryos. The flies of following genotypes were used for other experiments: tub-Gal4/+ (WT), AMPK^{D1}/Y; tub-Gal4/+(AMPK^{D1}), AMPK^{D1}/Y; UAS-AMPK^{WT}/tub-Gal4 (AMPK^{D1}, tub>AMPK^{WT}), and AMPK^{D1}/Y; UAS-AMPK^{KR}/tub-Gal4 (AMPK^{D1}, tub>AMPK^{KR}) for FIG. 1e; w¹¹⁸ (WT) and AMPK^{D1} (AMPK^{D1}) for FIG. 1f; hs-Gal4/+ (WT), AMPK^{D1}/Y; hs-Gal4/+ (AMPK^{D1}), AMPK^{D1}/Y; hs-Gal4/UAS-LKB1 (AMPK^{D1}, hs>LKB1), hs-Gal4/+; LKB1^{X5}/LKB1^{X5} (LKB1^{X5}), UAS-AMPK^{TD}/hs-Gal4; LKB1^{X5}/LKB1^{X5} (LKB1^{X5}), hs>AMPK^{TD}), AMPK^{D1}/Y; MRLC^{EE}/+ (AMPK^{D1}, MRLC^{EE}), and LKB1^{X5} MRLC^{EE}/LKB1^{X5} (LKB1^{X5}, MRLC^{EE}) for FIGS. 2 f-m, 3g-h, 8 and 11; w¹¹¹⁸ (WT), AMPK^{D1} (AMPK^{D1}), and LKB1^{X5} (LKB1^{X5}) for FIG. 2e.
Survival rate analysis. More than 200 first instar larvae of w¹¹¹⁸ and AMPK^{D1} mutants were cultured on sucrose-yeast medium and their viability was scored at each developmental stage. Green balancer chromosome (FM7 Act-GFP) was used to select the first instar larvae of homozygous AMPK^{D1} mutant.
Immunohistochemistry. Third-instar larvae were dissected in Drosophila Ringer's solution, and brains and imaginal discs were fixed in 4% formaldehyde phosphate-buffered saline (PBS) solution for 10 min at room temperature. Embryos were dechorionated by 50% bleach, fixed in 4% formaldehyde-PBS/heptane biphasic solution, devitelinized by methanol, and rehydrated in PBS. For the selection of LKB1-null homozygous embryos, blue balancer chromosome (TM3 hb-lacZ) was used, and X-Gal staining was performed before the devitelinization step (Lee et al., Cell Death Differ. 13, 110-1122 (2006)). After being washed with PBS-0.1% Triton X-100 (PBST), the samples were blocked for 1 hr at room temperature with PBST containing 5% bovine serum albumin (BSA). The samples were further incubated with the indicated antibodies at 4° C. for 16 hr (and with TRITC-labeled phalloidin to stain filamentous actins, if required). Following three 30 min washes in PBST, the samples were incubated with appropriate secondary antibodies for 3 hr (and with Hoechst 33258 to stain DNA, if required) at room temperature. The samples were washed with PBST and mounted in 80% glycerol-PBS solution, then observed with the confocal microscope LSM510 (Zeiss) (Lee et al., Cell Death Differ. 13, 110-1122 (2006)).
TUNEL staining. For TUNEL staining, embryos were collected, dechorionated, devitelinized, and rehydrated as described above. The samples were then permeablized by 4 μg/mL proteinase K in PBS for 10 min. After extensive washing, samples were submerged in TUNEL reaction solution (In Situ Cell Death Detection Kit, TMR red, Roche) then incubated at 37° C. for 3 hr. After rinsing 3 times with PBS, the samples were subjected to immunohistochemistry. For quantification of apoptotic cell death in FIG. 7g, we performed three replicate experiments (80-150 embryos were observed for each experiment) to calculate the proportion of the number of embryos with epithelial cell apoptosis to the total number of embryos. The average ratio of the three experiments is presented in % as a bar graph, and the standard deviation is indicated as error bars. A P-value was calculated using one-way ANOVA analysis.
Site-directed mutagenesis. For site-directed mutagenesis, QuickChange™ kit (Stratagene) was used. For generation of a kinase-dead mutant AMPK (Lys57Arg, AMPK^{KR}), 5′-GTCAAGGTGGCCGTCAGGATACTCAATCGTCAG-3′ and 5′-CTGACGATTGAGTATCCTGACGGCCACCTTGAC-3′ primers were used. For generation of an AMPK mutant non-phosphorylatable by LKB1 (Thr184Ala, AMPK^{TA}), 5′-CGAGTTCCTGCGCGCCTCGTGCGGCTC-3′ and 5′-GAGCCGCACGAGGCGC GCAGGAACTCG-3′ primers were used. For generation of an AMPK mutant mimicking LKB1-dependent phosphorylation (Thr184Asp, AMPK^{TD}), 5′-GCGAGTTC CTGCGCGACTCGTGCGGCTCGC-3′ and 5′-GCGAGCCGCACGAGTCGCGCAGG AACTCGC-3′ primers were used. As previously described^{S2}, amino acids 1-324 of AMPK^{TD} were subcloned into pUAST. For generation of a Thr18Ala-mutant form of human MRLC (hMRLC^{TA}), 5′-CCTCAGCGTGCAGCATCCAATGTGTTTGCT-3′ and 5′-AGCAAACACATTGGATGCTGCACGCTGAGG-3′ primers were used. For generation of a Ser19Ala-mutant form of human MRLC (hMRLC^{SA}), 5′-CCTCAGC GTGCAACAGCCAATGTGTTTGCT-3′ and 5′-AGCAAACACATTGGCTGTTGCA CGCTGAGG-3′ primers were used. For generation of a Thr18Ala/Ser19Ala-mutant form of human MRLC (hMRLC^{AA}), 5′-CCTCAGCGTGCAGCAGCCAATGTGTTTG CT-3′ and 5′-AGCAAACACATTGGCTGCTGCACGCTGAGG-3′ primers were used. For generation of a Thr18Glu/Ser19Glu-mutant form of human MRLC (hMRLC^{EE}), 5′-CGCCCTCAGCGTGCAGAAGAAAATGTGTTTGCTATG-3′ and 5′-CATAGCA AACACATTTTCTTCTGCACGCTGAGGGCG-3′ primers were used. For generation of a Thr21Ala/Ser22Ala-mutant form of Drosophila MRLC (dMRLC^{AA}), 5′-CGCGCCCAACGCGCCGCCGCCAATGTGTTCGCCATG-3′ and 5′-CATGGCGAA CACATTGGCGGCGGCGCGTTGGGCGCG-3′ primers were used.
Synthesis of double-stranded RNA. For synthesis of double-stranded RNA (dsRNA) of Drosophila AMPK and LKB1, we used oligonucleotides containing a T7 polymerase binding site (5′-TAATACGACTCACTATAGGG-3′) at the 5′ of the following primers, 5′-TTCGGCAAGGTGAAG-3′ and 5′-CACTTGCAGCATCTG-3′ (producing dsRNA from nucleotides 115 to 789 of the AMPK coding sequence) (Pan et al., Biochem. J. 367, 179-186 (2002)); 5′-GGATCTGTTCGC ACCTGACG-3′ and 5′-CTGCCGCTTGGCGGGCG-3′ (producing dsRNA from nucleotides 971 to 1479 of the LKB1 coding sequence) (Lee et al., Cell Death Differ. 13, 110-1122 (2006)). For synthesis of dsRNA to Drosophila Par-1 and MRLC, DRSC31375 and DRSC23800 amplicons were respectively amplified according to the manual of Drosophila RNAi Screening Center (DRSC). The purified PCR products were subjected to an in vitro T7 transcription reaction using the MEGAscript™ kit (Ambion). The complementary RNA strands were allowed to anneal together and were stored at −20° C.
Drosophila S2 cell culture. S2 cells were maintained at 25° C. in Shields and Sang M3 insect media (Sigma) with an insect medium supplement (Sigma). For dsRNA treatment, cells were diluted to a final concentration of 1×10⁶ cells/ml in M3 media. One milliliter of cells were plated per well of a six-well cell culture dish (SPL, Korea). 30 μg of dsRNA was directly added to the media. The cells were incubated for 3 days to allow for turnover of the targeted protein. One hour prior to stimulation, the culture media were replaced with fresh M3 media. The cells were then stimulated by 25 mM 2DG (Sigma) for 15 min (FIG. 5b) or 50 mM 2DG for 4 hr (FIG. 16). Appropriately treated cells were lysed with either Lysis Buffer [20 mM Tris-HCl (pH 7.5), 1% Triton X-100, 1 mM EDTA, 5 mM EGTA, 10 mM MgCl₂, 50 mM β-glycerophosphate, 1 mM sodium orthovanadate, 2 mM dithiothreitol, 40 μg/ml phenymethylsulfonyl fluoride, 1 μM peptistatin A, and 10 μg/ml leupeptin] (FIG. 5b) or SDS sample buffer [70 mM Tris-HCl (pH 6.8), 2% glycerol, 0.002% bromophenolblue, 3% SDS, and 5% β-mercaptoethanol] (FIG. 16). The lysates were subjected to immunoblot analyses. Immunoblot using anti-LKB1 antibody was performed after immunoprecipitation (Kim et al., EMBO J. 25, 3056-3067 (2006)) using the same antibody. For the preparation of Drosophila AMPK, pRmHa-HA-AMPK^{WT} and pRmHa-HA-AMPK^{KR}, which were subcloned from pUAST constructs, were transfected into S2 cells via the DDAB method (Han et al., Nucleic Acids Res. 24, 4362-4363 (1996)). pRmHa empty vector was obtained from Drosophila genomics resource center (DGRC).
Immunoblot. Lysates were boiled in SDS sample buffer (described above) for 10 min. The samples were subjected to SDS-PAGE and proteins were transferred to nitrocellulose membranes (Schleicher & Schuell, #BA83 was used for detection of pMRLC or MRLC protein, and #BA85 was used for detection of all other proteins). Membranes were boiled in PBS for 5 min, and incubated for 30 min in Blocking Solution [Tris-buffered saline (TBS) containing 0.1% Tween-20, 5% BSA and 0.02% sodium azide] and further incubated with the appropriate primary antibody at 4° C. overnight. The membranes were then washed four times with 0.1% Tween-20/TBS and incubated for 30 min with secondary antibody conjugated to horseradish peroxidase (Amersham). Bound antibodies were detected with enhanced chemiluminescence.
Preparation of AMPK enzyme. For the experiments in FIGS. 3 a-d and 12, AMPK holoenzyme purified from rat liver was purchased from Upstate (#14-305). For the experiments in FIGS. 13a and 13c, Drosophila AMPK was purified from heat shock-treated hs-Gal4/+, hs-Gal4/UAS-AMPK^{WT}, hs-Gal4/UAS-AMPK^{KR}, and hs-Gal4/UAS-AMPK^{TA} flies (each form of AMPK was tagged with HA epitope, as described above). For the experiments in FIGS. 13b, 14 a-b, Drosophila AMPK was purified from S2 cells transfected with pRmHa-HA-AMPK^{WT} or pRmHa-HA-AMPK^{KR}. For the experiments in FIGS. 13a, b, d, and 14a-b, human AMPK was purified from HE 293T cells transfected with pcDNA3-HA-AMPKWT or pcDNA3-HA-AMPKDN (kindly provided by Dr. K. L. Guan). For the experiments in FIG. 13a-b and 14a, S2 cells transfected with pRmHa empty vector and HEK293T cells transfected with pcDNA3 empty vector (Invitrogen) were used as controls. For the experiments in FIG. 14b, active MLCK was purchased from Upstate (#14-638). For the experiments in FIG. 14c, GST-tagged human AMPK alpha was purchased from Cell Signaling (#7464). For the purification of HA-tagged AMPK, the immunoprecipitation method was used as previously described (Kim et al., EMBO J. 25, 3056-3067 (2006)). In detail, samples were homogenized in Lysis Buffer (described above). The lysates were clarified by centrifugation at 14,000 rpm for 10 min at 4° C. HA-tagged AMPK enzyme was immunoprecipitated by anti-HA antibody (12CA5, Roche) coupled to protein G-Sepharose beads (Amersham). The enzyme-bead complexes were washed twice with Lysis Buffer, then twice with Lysis Buffer containing 500 mM NaCl, and finally with HEPES-Brij Buffer [50 mM Na-HEPES (pH 7.4), 1 mM DTT, 5 mM MgCl₂, and 0.02% Brij-35]. The purified enzyme-bead complexes were subjected to in vitro kinase assay or silver staining. Silver staining was performed with Silver Stain Plus (Bio-Rad) according to the manufacturer's instruction.
Preparation of kinase assay substrates. GST-ACC2 was purchased from Upstate (#12-491). Drosophila MRLC cDNA obtained from DGRC and human MRLC cDNA amplified from human keratinocyte cDNA library (Koh et al., Oncogene 18, 5115-5119 (1999)) were cloned into pGEX 4T-1 vector (Amersham). In addition, mutant forms of MRLC were generated by site-directed mutagenesis, as described above. Proteins were expressed in E. coli (BL21, Invitrogen), and purified using GSH-agarose column (Peptron, Korea), according to the manufacturer's instruction. For kinetic analyses in FIG. 3c, each substrate was concentrated in HEPES-Brij Buffer (described above) using the YM-10 Microcon device (Millipore).
In vitro kinase assay—P81 filter binding assay. In detail, the assay samples were transferred to P81 phosphocellulose filters (2 cm×2 cm squares; Whatman) for total ³²P incorporation of the kinase and substrate. Filters were washed five times with 0.75% phosphoric acid for 5 min and the filters were dehydrated using acetone. The amount of incorporated ³²P was measured by liquid-scintillation counting. The specific activity of ATP was determined by spotting an aliquot of reaction mixture containing 1 pmol ATP onto a P81 filter, and counting directly without washing. Counts per minute obtained in the kinase reaction are then divided by the specific activity to determine the moles of phosphate transferred in the reaction. The amount of phosphates transferred to substrate was determined by subtracting ³²P incorporation of AMPK from total ³²P incorporation of AMPK plus substrate.
Mammalian cell culture and transfection. LS174T cells were grown in RPMI 1640 media (Invitrogen) and HEK293T cells were grown in DMEM media (Invitrogen) supplemented with 10% fetal bovine serum (Invitrogen) at 37° C. in a humidified atmosphere of 5% CO₂. For AMPK activation, cells were stimulated with 50 mM 2DG (Sigma). One hour prior to stimulation, the culture media were replaced with fresh serum-free media. The cells were then treated by 2DG for the indicated times. 10 μM Compound C (Calbiochem) was treated for 30 min before 2DG stimulation to inhibit AMPK activity. pcDNA3, pcDNA3-HA-hAMPK, pcDNA3-HA-hAMPK^{DN} (described above), pCS2+Wnt1 (kindly provided by Dr. R. T. Moon), pEBG, pEBG-HA-Par-1^{DN} (modified from the pCMV-HA-Par-1, which was kindly provided by Dr. H. Piwnica-Worms), pEGFP-N1 (Clonetech), or pEGFP-N1-MRLC^{EE} was transfected to LS174T and HEK293T cells using lipofectamine plus reagent (Invitrogen). Dominant-negative effects of AMPK^{DN} in pcDNA3-HA-hAMPK^{DN} vector have been previously reported (Inoki et al., Cell 115, 577-590 (2003)). Dominant-negative effects of Par-1^{DN} in pEBG-HA-Par-1^{DN} vector were confirmed by luciferase assay as previously described^{S6}, using a dual luciferase reporter assay kit (Promega) according to the manufacturer's instructions. Control siRNA (D-001210-01-20), Par-1 siRNA (M-003517-03) and MRLC siRNA (M-018116-00) were purchased from Dharmacon, and were transfected by the XtremeGENE siRNA Transfection Reagent (Roche) according to the manufacturer's instruction. For RT-PCR of human actin, 5′-CGTACCACTGGCATCGTGAT-3′ and 5′-GTGTTGGCGTACAGGTCTTT-3′ primers were used. For RT-PCR of human Par-1,5′-CTCAGTTGAATCCAACAAG TCTAC-3′ and 5′-CTCCACTTGCATATTCCATGATTAG-3′ primers were used. For quantification of actin polarization in FIGS. 20 f-h, we calculated the proportions of the number of cells with actin cap formation to the total number of cells counted, and presented them as bar graphs, as previously described (Baas et al., Cell 116, 457-466 (2004)). For the transfection experiments, transfected cells were identified by anti-HA immunostaining or GFP fluorescence. The total number of cells counted is indicated in the legend to the Figures.
Immunocytochemistry. For immunocytochemistry, LS174T cells were sub-cultured on μ-Slide 8-well (Ibidi) coated with poly-L-lysine (Sigma). Appropriately treated cells were washed three times with cold PBS, and were fixed in 2% paraformaldehyde for 15 min, followed by permeabilization in 0.5% Triton X-100 for 5 min. Then, the cells were incubated in blocking solution (3% BSA and 1% normal goat serum in PBST) for 1 hr. Primary antibodies were added to the blocking solution and the cells were incubated for 1 hr at 37° C. After washing with PBST three times, the cells were incubated with appropriate secondary antibodies in blocking solution for 45 min at room temperature. Then, the cells were labeled by 66 nM TRITC-labelled phalloidin (Sigma) in PBST for 10 min at room temperature. The labeled cells were washed with PBST six times and were mounted with mounting solution (100 mg/ml 1,4-diazabicyclo[2.2.2]octane (DABCO) in 90% glycerol). The slides were observed with a LSM510 laser scanning confocal microscope (Zeiss).
Electron microscopy of LS174T cells. For transmission electron microscopy (TEM), cells were sub-cultured on poly-L-lysine (Sigma) coated 100 mm dish. Cultured cells were fixed with 3% glutaraldehyde in culture medium for 2 hr at room temperature. They were washed five times with 0.1 M cacodylate buffer containing 0.1% CaCl₂ at 4° C. Then, they were postfixed with 1% OSO₄ in 0.1 M cacodylate buffer (pH 7.2) containing 0.1% CaCl₂ for 2 hr at 4° C. After rinsing with cold distilled water, cultured cells were scratched out from the petri dish by cell scraper and transferred into micro-centrifuge tubes at 4° C. Cells were collected by centrifugation and embedded in 1% ultra-low gelling temperature agarose (Sigma, type IX). These cells were dehydrated slowly with an ethanol series and propylene oxide at 4° C. The cells were embedded in Spurr's epoxy resin (Spurr, J. Ultrastruct. Res. 26, 31-43 (1969)). After polymerization of the resin at 70° C. for 36 hr, serial sections were cut with a diamond knife on an ULTRACUT UCT ultramicrotome (Leica) and mounted on formvar-coated slot grids. Sections were stained with 4% uranyl acetate for 10 min and lead citrate (Reynolds, J. Cell Biol. 17, 208-212 (1963)) for 7 min, and observed by a Tecnai G2 Spirit Twin transmission electron microscope (FEI company) and JEM ARM 1300S high-voltage electron microscope (JEOL, Japan). For scanning electron microscopy (SEM), cells were sub-cultured on poly-L-lysine (Sigma) coated coverslips. Appropriately treated cells were fixed in 2.5% paraformaldehyde-glutaraldehyde mixture buffered with PBS (pH 7.2) for 2 hr, postfixed in 1% osmium tetroxide in the same buffer for 1 hr, dehydrated in graded ethanol, and substituted by isoamyl acetate. Then, they were dried at the critical point in CO₂. Finally the samples were sputtered with gold in a sputter coater (SC502, Polaron) and observed using the scanning electron microscope, LEO 1455VP.

We claim:

1. A transgenic Drosophila embryo comprising in its germ cells an adenosine monophosphate-activated protein kinase (AMPK) null mutation on both AMPK alleles, wherein said mutation results in said embryo exhibiting, compared to a Drosophila embryo lacking said null mutation, at least one phenotype selected from the group consisting of
a) increase in number of embryos that do not develop into larvae,
b) change in cuticle structure,
c) decrease in number of ventral denticle belts,
d) change in organization of epidermis tissue,
e) change in epithelial cell polarity,
f) decrease in number of embryos forming a cuticle,
g) decrease in level of expression around an epithelial basolateral surface of at least one of apical complex marker and of β-catenin,
h) increase in number of unpolarized round epithelial cells lacking contact with underlying tissue,
i) increase in number of ectopic actin structures in a basolateral region of a wing disc,
j) increase in nuclear size,
k) change in metaphase chromosome alignment,
l) increase in lagging chromosomes during anaphase,
m) increase in chromosomal polyploidy in a cell, and
n) increase in chromosome content in a brain neuroblast cell.

2. The transgenic Drosophila embryo of claim 1, wherein said cell having chromosomal polyploidy comprises a brain neuroblast cell.

3. The transgenic Drosophila embryo of claim 1, wherein epithelial cells of said embryo comprise reduced levels of phosphorylated non-muscle myosin regulatory light chain (MRLC) compared to a Drosophila embryo lacking said null mutation.

4. The transgenic Drosophila embryo of claim 1, wherein increased expression of non-muscle myosin regulatory light chain (MRLC) in said embryo results in at least one phenotype selected from the group consisting of
a) reversal of said change in said epithelial cell polarity,
b) increase in number of embryos that form a cuticle, and
c) decrease in chromosomal polyploidy.

5. The transgenic Drosophila embryo of claim 1, wherein said embryo is generated by mating a male transgenic Drosophila and a female transgenic Drosophila each bearing one artificially mutated AMPK allele in its germ cells.

6. The transgenic Drosophila embryo of claim 5, wherein said artificially mutated AMPK allele is generated by an autosomal flipase recombination target dominant female sterile (FLP-DFS) method.

7. A method for screening a drug candidate for treatment of a disease comprising
a) providing
i) the transgenic Drosophila embryo of claim 1, and
ii) a drug candidate,
b) administering said drug candidate to said embryo, and
c) determining said embryo's response to said drug candidate.

8. The method of claim 7, wherein said response comprises a change in at least one of said phenotypes.

9. The method of claim 7, wherein said disease is selected from the group consisting of cancer, kidney disease, diabetes, intestinal disease, and obesity.

10. The method of claim 7, wherein said disease comprises increased body weight of said subject.

11. The method of claim 7, wherein said drug candidate is an AMPK-activating drug.

12. The method of claim 11, wherein said AMPK-activating drug comprises one or more of metformin (N,N-dimethylimidodicarbonimidic diamide hydrochloride), AICAR (5-aminoimidazole-4-carboxamide-1-β-D-ribofuranoside), resveratrol (trans-3,4′,5-trihydroxystilbene), and a thiazolidinedione compound.

13. The method of claim 11, wherein said disease is selected from the group consisting of cancer, kidney disease, diabetes and intestinal disease.

14. The method of claim 13, wherein said intestinal disease is characterized by reduced nutrient absorption by intestines.

15. The method of claim 13, wherein said intestinal disease is caused by an organism that alters intestinal epithelial cell polarity.

16. The method of claim 15, wherein said organism comprises Salmonella typhimurium.

17. The method of claim 7, wherein said drug candidate is an AMPK-inhibiting drug.

18. The method of claim 17, wherein said AMPK-inhibiting drug comprises compound C.

19. The method of claim 17, wherein said disease comprises increased body weight.

20. The method of claim 19, wherein said increased body weight comprises obesity.

21. The method of claim 17, wherein said disease comprises kidney disease.

22. A method for detecting a disease in a tissue, comprising detecting a change in AMPK activity in said tissue compared to a control tissue.

23. The method of claim 22, wherein said disease is selected from the group consisting of cancer, kidney disease, diabetes, and intestinal disease.

24. A method for reducing symptoms of a disease in a subject, comprising administering a therapeutic amount of a drug that changes AMPK activity to said subject.

25. The method of claim 24, further comprising determining a reduction in symptoms of said disease.

26. The method of claim 24, wherein said change in AMPK activity is a reduction in AMPK activity, and said disease is selected from the group consisting of kidney disease and increased body weight.

27. The method of claim 24, wherein said change in AMPK activity is an increase in AMPK activity, and said disease is selected from the group consisting of kidney disease and intestinal disease.
